(19) United States
(12) Patent Application Publication
(10) Pub. No.: US 2024/0200112 A1
(43) Pub. Date: Jun. 20, 2024

(54) Process for the Production of Fucosylated Oligosaccharides

(71) Applicant: Chr. Hansen HMO GmbH, Rheinbreitbach (DE)

(72) Inventors: Stefan Jennewein, Bad Honnef (DE); Dirk Wartenberg, Bonn (DE); Katja Parschat, Bonn (DE)

(73) Assignee: Chr. Hansen HMO GmbH, Rheinbreitbach (DE)

(21) Appl. No.: 18/393,339

(22) Filed: Dec. 21, 2023

Related U.S. Application Data
(63) Continuation of application No. 16/345,535, filed on Apr. 26, 2019, now Pat. No. 11,898,185.

(30) Foreign Application Priority Data
Oct. 29, 2016 (EP) 16196486.1

(51) Int. Cl. C12P 19/18; C07K 14/24; C12N 9/04; C12N 9/10; C12N 9/12; C12N 9/16; C12N 9/88; C12N 9/90; C12N 15/52; C12N 15/70

(52) U.S. Cl. CPC C12P 19/18; C07K 14/24; C12N 9/0006; C12N 9/1051; C12N 9/12; C12N 9/1205; C12N 9/1241; C12N 9/16; C12N 9/88; C12N 9/90; C12N 15/52; C12N 15/70; C12Y 101/01271; C12Y 204/01065; C12Y 207/01011; C12Y 207/07013; C12Y 301/03011; C12Y 401/02013; C12Y 402/01047; C12Y 504/02008

(57) ABSTRACT

The present invention relates to a method for producing fucosylated oligosaccharides by using a recombinant prokaryotic host cell that is cultivated on a gluconeogenic substrate, as well as to the host cell and its use. The host cell is genetically modified in that the activity of a fructose-6-phosphate converting enzyme is abolished or lowered, and the transport of the produced fucosylated oligosaccharide through the cell membrane is facilitated by an exogenous transport protein.

### CROSS-REFERENCE TO RELATED APPLICATIONS

This application is a Continuation of U.S. application Ser. No. 16/345,535 filed Apr, 26, 2019, which is a National Stage entry of International Application No. PCT/EP2017/077192, filed 24 Oct. 2017, which claims priority to European Patent Application No. 16196486.1, filed 29 Oct. 2016, the entire contents of which are incorporated by reference in their entireties.

### SEQUENCE LISTING

The present application contains a Sequence Listing which has been submitted electronically. Said copy, created on Dec. 19, 2023, is named “2023-12-19_01314-0006-01US_ST26.txt” and is 35,382 bytes in size. The information in the electronic format of the sequence listing is incorporated herein by reference in its entirety.

The present invention relates to a method for the production of fucosylated oligosaccharides using a genetically modified prokaryotic host cell, as well as to a host cell employed in this method and its use for producing fucosylated oligosaccharides to high titers.

### BACKGROUND OF THE INVENTION

Human milk represents a complex mixture of carbohydrates, fats, proteins, vitamins, minerals and trace elements. The by far most predominant fraction is represented by carbohydrates, which can be further divided into lactose and more complex oligosaccharides (Human milk oligosaccharides, HMO). Whereas lactose is used as an energy source, the complex oligosaccharides are not metabolized by the infant. The fraction of complex oligosaccharides accounts for up to 20% of the total carbohydrate fraction and consists of more than 200 different oligosaccharides. The occurrence and concentration of these complex oligosaccharides are specific to humans and thus cannot be found in large quantities in the milk of other mammals.

Approximately 200 structurally-diverse HMOs have been identified so far and numerous beneficial properties have been reported. HMOs are not digested by breastfed infants, but represent a valuable carbon and energy source for beneficial bacteria of the genera Bifidobacteria, Lactobacillus and Bacteroides in the intestine, causing them to become dominant in the gut and allowing them to outcompete pathogens, thus preventing infections of the gut epithelium. However, HMOs also bind directly to pathogenic bacteria, protozoa and viruses, blocking pathogen-host interactions by mimicking glycan cell surface receptors and thereby protecting the breastfed child from infectious diseases.

The most prominent oligosaccharide is 2′-fucosyllactose. Further prominent HMOs present in human milk are 3-fucosyllactose, lacto-N-tetraose, lacto-N-neo-tetraose and the lacto-N-fucopentaoses. Besides these neutral oligosaccharides, also acidic HMOs can be found in human milk, like for e.g. 3′-sialyllactose, 6′-sialyllactose and sialyllacto-N-tetraose a, b, and c, or sialyllacto-N-fucpentaose II etc. These structures are closely related to epitopes of epithelial cell surface glycoconjugates, the Lewis histoblood group antigens, and the structural homology of HMO to epithelial epitopes accounts for protective properties against bacterial pathogens.

Due to their beneficial properties, HMOs are favored for inclusion as ingredients in infant formulae and other food products, necessitating the production of HMOs in large quantities, up to the multi-ton scale.

Due to the limited supply and difficulties of obtaining pure fractions of individual human milk oligosaccharides, chemical routes to some of these complex molecules were developed. However, chemical and biocatalytic approaches proved to be not commercially sustainable, and, furthermore, particularly chemical synthetic routes to human milk oligosaccharides involve several noxious chemicals, which impose the risk to contaminate the final product.

Due to the challenges involved in the chemical synthesis of human milk oligosaccharides, several enzymatic methods and fermentative approaches were developed. Today, for several HMOs such as 2′-fucosyllactose, 3-fucosyllactose, lacto-N-tetraose, lacto-N-neotetraose, lacto-N-fucopentaose I, lacto-N-difucohexaose II, 3′-sialyllactose and 6′-sialyllactose fermentative approaches have been developed, using mainly genetically engineered bacterial strains such as recombinant Escherichia coli.

However, even the most efficient processes based on bacterial fermentation, which are available today, do not or hardly achieve HMO titers greater than 20 g/L in the culture broth. Industrial-scale processes must typically exceed titers of 50 g/L although 100 g/L is more desirable.

Thus, it is an object of the present invention to provide for an improved fermentation process by means of which a biosynthesis of fucosylated oligosaccharides, in particular of 2′-fucosyllactose, is enabled at a titer exceeding more than 100 g/L.

### SUMMARY OF THE INVENTION

This, and other objects are solved by a method for the production of fucosylated oligosaccharides using a genetically modified prokaryotic host cell, the method comprising the steps of:
providing a host cell, which has been genetically modified, such, that at least (i) the activity of a fructose-6-phosphate-converting enzyme, which in the unmodified host cell has a regular level, is lowered or abolished; (ii) at least one gene encoding an enzyme necessary for the de novo synthesis of GDP-fucose is overexpressed in the host cell; (iii) an exogenous gene, encoding a fucosyltransferase, preferably an alpha-1,2-fucosyltransferase and/or alpha-1,3-fucosyltransferase, is expressed, preferably overexpressed, in the host cell;
cultivating said genetically modified host cell in a cultivation medium containing a carbon and energy source that is selected from at least one of the following: glucose, sucrose, glycerol, succinate, citrate, pyruvate, malate, lactate, or ethanol; and
providing lactose to the cultivation medium with lactose.

In a subsequent step, the thus produced fucosylated oligosaccharide may be retrieved or obtained from the medium the host cell is cultivated in.

The step of growing and cultivating the genetically modified host cell and the step of adding lactose to the cultivation medium can be performed such, that the genetically modified host cell is first cultivated for a certain period of time, and, in a subsequent step after this first cultivation time, lactose is provided by adding it to the medium the host cell is cultivated in; alternatively, lactose may be provided from the beginning of the cultivation time of the genetically modified host cell, in a certain amount, and may be constantly added in a certain amount. Alternatively, lactose can be generated internally.

The object is further solved by a genetically modified prokaryotic host cell and by its use for the production of a fucosylated oligosaccharide, which host cell has been genetically modified such, that at least (i) the activity of a fructose-6-phosphate-converting enzyme, which in the unmodified host cell is at a regular level, is lowered or abolished and/or by having increased the activity of a fructose-6-phosphate generating enzyme in the host cell; (ii) at least one gene encoding an enzyme necessary for the de novo synthesis of GDP-fucose is overexpressed; (iii) an exogenous gene encoding a fucosyltransferase, preferably an alpha-1,2-fucosyltransferase and/or alpha-1,3-fucosyltransferase, is expressed, preferably overexpressed, in the host cell.

Optionally, the host cell has been genetically further modified (iv) to express a gene encoding a protein enabling or facilitating the transport of the desired fucosylated oligosaccharide into the medium the host cell is cultivated in; and/or (v) to express an exogenous gene encoding a bifunctional L-fucokinase/L-fucose 1-phosphate guanylyltransferase; and/or (vi) to have inactivated or deleted genes encoding a L-fucose-isomerase and L-fuculose-kinase; and/or (vii) to have inactivated or disrupted genes coding for enzymes of the colanic acid synthesis; and/or (viii) to express a lactose permease; and/or (ix) to have inactivated or deleted endogenous beta-galactosidase genes; and/or (x) to express an exogenous regulable beta-galactosidase gene; and/or (xi) to overexpress exogenous genes for metabolizing galactose, and/or (xii) to express an exogenous gene encoding an enzyme exhibiting fructose-1,6-bisphosphate phosphatase activity.

Also, herein provided is a method for the production of fucosylated oligosaccharides using a genetically modified prokaryotic host cell, the method comprising the steps of:
providing a prokaryotic host cell, which has been genetically modified, such, that at least (i) the fructose-6-phosphate pool in said genetically modified host cell is increased by having lowered or abolished the activity of a fructose-6-phosphate-converting enzyme, which in the unmodified host cell has a regular level, or by having increased the activity of a fructose-6-phosphate generating enzyme; that (ii) at least one gene encoding an enzyme necessary for the de novo synthesis of GDP-fucose is overexpressed in the host cell; (iii) an exogenous gene encoding an alpha-1,2-fucosyltransferase and/or alpha-1,3-fucosyltransferase is expressed in the host cell;
cultivating said genetically modified host cell in a cultivation medium containing a carbon and energy source that is selected from at least one of the following: glucose, sucrose, glycerol, succinate, citrate, pyruvate, malate, lactate, or ethanol; and
providing lactose to the cultivation medium with lactose.

The objects underlying the invention are completely solved in this way.

With the method according to the invention, as well as with the genetically modified host cell employed in the method, it is possible to produce fucosylated oligosaccharides at a titer exceeding 50 g/L, and even 100 g/L, and even more 150 g/L, thus providing a successful tool for the large-scale and, thus, industrial-scale fermentative production of fucosylated oligosaccharides.

Presently, and as generally understood in the state of the art, a “fucosylated oligosaccharide” is a fucosylated oligosaccharide as found in human milk, i.e. an oligosaccharide that is carrying a fucose-residue. Preferably, the fucosylated oligosaccharide is one that is selected from 2′-fucosyllactose, 3-fucosyllactose or difucosyllactose.

Also, a “genetically modified prokaryotic host cell” presently means a prokaryotic cell whose genetic material has been altered using genetic engineering techniques. E.g., the host cell has been genetically modified, such, that either endogenous nucleic sequences naturally occurring in said host cell have been deleted, interrupted or otherwise influenced so that their expression is modified, i.e. abolished, lowered, suppressed, enhanced, or similar, and/or exogenous nucleic acids, i.e. nucleic acids that are foreign to said host cell, have been introduced into the host cell to be expressed, e.g. under control of an controllable promoter, in the host cell. In this connection, such genetically modified host cells are also called “recombinant host cells”. For example, a subject prokaryotic host cell is a genetically modified prokaryotic host cell, by virtue of introduction into a suitable prokaryotic host cell a heterologous nucleic acid, e.g., an exogenous nucleic acid that is foreign to the prokaryotic host cell, or a recombinant nucleic acid that is not normally found in the prokaryotic host cell.

Accordingly, the term “recombinant”, as used herein with reference to a bacterial host cell indicates that the bacterial cell replicates a heterologous nucleic acid, or expresses a peptide or protein encoded by a heterologous nucleic acid (i.e., a sequence “foreign to said cell”). Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. Accordingly, a “recombinant polypeptide” is one which has been produced by a recombinant cell. A “heterologous sequence” or a “heterologous nucleic acid”, as used herein, is one that originates from a source foreign to the particular host cell (e.g. from a different species), or, if from the same source, is modified from its original form. Thus, a heterologous nucleic acid operably linked to a promoter is from a source different from that from which the promoter was derived, or, if from the same source, is modified from its original form. The heterologous sequence may be stably introduced, e.g. by transfection, transformation, conjugation or transduction, into the genome of the host microorganism cell, wherein techniques may be applied which will depend on the host cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989).

Accordingly, a “genetically modified prokaryotic host cell” is presently understood as a prokaryotic cell which has been transformed or transfected, or is capable of transformation or transfection by an exogenous polynucleotide sequence.

The nucleic acid sequences as used in the present invention, may, e.g., be comprised in a vector which is to be stably transformed/transfected or otherwise introduced into host microorganism cells.

A great variety of expression systems can be used to express the genes in the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and to synthesize a polypeptide in a host may be used for expression in this regard. The appropriate DNA sequence may be inserted into the expression system by any of a variety of well-known and routine techniques, such as, for example, those set forth in Sambrook et al., supra.

The art is rich in patent and literature publications relating to “recombinant DNA” methodologies for the isolation, synthesis, purification and amplification of genetic materials for use in the transformation of selected host organisms. Thus, it is common knowledge to transform host organisms with “hybrid” viral or circular plasmid DNA which includes selected exogenous (i.e. foreign or “heterologous”) DNA sequences. A person skilled in the art will know a variety of methods to achieve “hybrid” vectors for use in the transformation of a selected host organism.

The term “nucleic acid sequence encoding . . . ” generally refers to any polyribonucleotide or polydeoxyribonucleotide, which may be unmodified RNA or DNA or modified RNA or DNA, and generally represents a gene which encodes a certain polypeptide or protein. The term includes, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

As used herein, the term “cultivating” means growing and/or incubating a bacterial cell in a medium and under conditions permissive and suitable for the production of the desired oligosaccharide(s). A couple of suitable bacterial host cells as well as mediums and conditions for their cultivation will be readily available for one skilled in the art upon reading the disclosure of this invention in connection with the skilled person's technical and expert background.

It is to be understood that with the invention as disclosed herein, the production of one or more oligosaccharides as defined herein is possible, as long as the respective nucleic acids encoding the relevant proteins/enzymes as disclosed herein are comprised in the cell(s).

As used herein, the term “recovering” means isolating, harvesting, purifying, collecting or otherwise separating from the host microorganism culture the oligosaccharide produced by the host microorganism according to the invention.

According to an embodiment of the method and the use of the invention, the fucosylated oligosaccharide to be produced is selected from at least one of 2′-fucosyllactose, 3-fucosyllactose or difucosyllactose.

According to an embodiment of the method of the invention, the prokaryotic host cell is selected from the group consisting of bacterial host cells, preferably selected from an Escherichia coli strain, a Lactobacillus species, or a Corynebacterium glutamicum strain.

Preferably, the host cell is a bacterial host cell selected from an Escherichia coli, Corynebacterium glutamicum, Bacillus subtilis, Bacillus megaterium, Lactobacillus casei, Lactobacillus acidophilus, Lactobacillus helveticus, Lactobacillus delbrueckii, Lactococcus lactis cell. A person skilled in the art will be aware of further bacterial strains when reading the present disclosure.

Presently, and as generally understood, the expression “fucosyltransferase” is understood as an enzyme that transfers an L-fucose sugar from a GDP-fucose (guanosine diphosphate-fucose) donor substrate to an acceptor substrate to form the fucosylated oligosaccharide. The acceptor substrate, in the present invention, is an oligosaccharide. Also, fucosyltransferases not only catalyze fucosylation in the presence of glycan acceptors, but can also hydrolyze GDP-L-fucose when no acceptor substrate is available.

Accordingly, the terms “alpha-1,2-fucosyltransferase” or “fucosyltransferase” or a nucleic acid/polynucleotide encoding an “alpha-1,2-fucosyltranferase” or “fucosyltransferase” refer to a glycosyltransferase that catalyzes the transfer of the fucose moiety from a donor substrate, for example, GDP-fucose, to an acceptor molecule in an alpha-1,2-linkage. The terms “alpha-1,3-fucosyltranferase” or “fucosyltransferase” or a nucleic acid/polynucleotide encoding an “alpha-1,3-fucosyltranferase” or “fucosyltransferase” refer to a glycosyltransferase that catalyzes the transfer of the fucose moiety from a donor substrate, for example, GDP-fucose, to an acceptor molecule in an alpha-1,3-linkage. The acceptor molecule can be, e.g., lactose, 2′-fucosyllactose, 3-fucosyllactose, 3′-sialyllactose, 6′-sialyllactose, lacto-N-tetraose, lacto-N-neotetraose or a derivative thereof.

According to the invention, the exogenous gene encoding a fucosyltransferase is selected from a gene expressing a protein exhibiting an alpha-1,2-fucosyltransferase activity, a gene expressing a protein exhibiting an alpha-1,3-fucosyltransferase activity, or a gene expressing an alpha-1,2-fucosyltransferase as well as an alpha-1,3-fucosyltransferase activity.

According to preferred embodiments, for the synthesis of 2′-fucosyllactose a suitable alpha-1,2-fucosyltransferase is expressed, for the synthesis of 3-fucosyllactose a suitable alpha-1,3-fucosyltransferase is expressed, for the synthesis of 2′,3-difucosyllactose, both, a suitable alpha-1,2-fucosyltransferase and an alpha-1,3-fucosyltransferase or at least one gene encoding for a protein exhibiting an alpha-1,2- as well as an alpha-1,3-fucosyltransferase activity is expressed.

Non-limiting examples for fucosyltransferases which can be used according to the invention and which shall be part of the invention are, e.g., bacterial fucosyltransferases, and preferably an alpha-1,2-fucosyltransferase, and more preferably the alpha-1,2-fucosyltransferase encoded by the wbgL gene of E. coli:O126, or the alpha-1,2-fucosyltransferase encoded by the fucT gene of Helicobacter pylori, or an alpha-1,3-fucosyltransferase, and more preferably an alpha-1,3-fucosyltransferase from Akkermansia muciniphila , Bacteroides fragilis, H. pylori, or H. hepaticus. Preferably, a glycosyl-transferase is used or variants thereof which is disclosed in EP 2 479 263 A1 or from EP 2 439 264 or in WO 2010/142305, the content of which is herewith explicitly referred to and made subject matter of this invention.

A “variant” as the term is used herein, is a polynucleotide or polypeptide that differs from a reference polynucleotide or polypeptide, in particular an enzyme as mentioned and used herein, respectively, but retains the essential (enzymatic) properties of the reference polynucleotide or polypeptide. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

Within the scope of the present invention, also nucleic acid/polynucleotide and polypeptide polymorphic variants, alleles, mutants, and interspecies homologs are comprised by those terms, that have an amino acid sequence/nucleic acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide as mentioned herein, e.g. to an fructose-6-phosphate-converting enzyme, in particular phosphofructokinase A, glucose-6-phosphate isomerase, fructose-6-phosphate aldolase, a transketolase, e.g. tktA, tktB, or a transaldolase, e.g. talA, talB, a fructose-1,6-bisphosphate phosphatase, as used herein, to the phosphomannomutase, preferably manB, a mannose-1-phosphate guano-syltransferase, preferably manC, a GDP-mannose-4,6-dehydratase, preferably gmd, and a GDP-L-fucose synthase, preferably wcaG, to a fucosyltransferase as used herein, to a fructose-1,6-bisphosphate phosphatase, preferably the functional active variant of the fructose-1,6-bisphosphate phosphatase (fbpase) from Pisum sativum, to the sugar efflux transporter, e.g. yberc0001_9420 or SetA, and to the lactose permease, e.g. LacY, used herein.

Accordingly, a “functional fragment” of any of the genes/proteins disclosed therein, is meant to designate sequence variants of the genes/proteins still retaining the same or somewhat lesser activity of the gene or protein the respective fragment is derived from.

As defined herein, the term “endogenous” herein and generally within the field means that the nucleic acid encoding for an enzyme of interest is originating from the bacterial host cell and has not been introduced into said host cell, whereas an “exogenous” or “recombinant” nucleic acid has been introduced into said host cell and does not originate from said host cell.

According to another embodiment, the nucleic acid/gene is homologous or heterologous. Presently, and as generally understood in the relevant field, the expression “homologous” refers to a nucleic acid sequence/gene that encodes for a specific product or products and is derived from the same species, in which said nucleic acid sequence is inserted. Accordingly, the term “heterologous” refers to a nucleic acid sequence/gene encoding for a specific product or products and being derived from a species other than those in which said nucleic acid sequence/gene is inserted.

According to another embodiment, the host cell of the invention further comprises control sequences enabling the controlled overexpression of endogenous or exogenous/recombinant nucleic acid sequences/genes. As defined above, the term “control sequence” which herein is synonymously used with the expression “nucleic acid/gene expression control sequence”, comprises promoter sequences, signal sequence, or array of transcription factor binding sites, which sequences affect transcription and/or translation of a nucleic acid sequence or gene operably linked to the control sequences.

Presently, the term “operably linked” as used herein, shall mean a functional linkage between a nucleic acid/gene expression control sequence (such as a promoter, signal sequence, or array of transcription factor binding sites) and a second nucleic acid sequence or gene, wherein the expression control sequence affects transcription and/or translation of the nucleic acid corresponding to the second sequence. Accordingly, the term “promoter” designates DNA sequences which usually “precede” a gene in a DNA polymer and provide a site for initiation of the transcription into mRNA. “Regulator” DNA sequences, also usually “upstream” of (i.e., preceding) a gene in a given DNA polymer, bind proteins that determine the frequency (or rate) of transcriptional initiation. Collectively referred to as “promoter/regulator” or “control” DNA sequence, these sequences which precede a selected gene (or series of genes) in a functional DNA polymer cooperate to determine whether the transcription (and eventual expression) of a gene will occur. DNA sequences which “follow” a gene in a DNA polymer and provide a signal for termination of the transcription into mRNA are referred to as transcription “terminator” sequences.

As outlined already further above, the nucleic acid sequence/gene which is used according to the invention, may, e.g., be comprised in a vector which is to be stably transformed/transfected into bacterial host cells. The definitions and detailed description for recombinant production as outlined above shall apply for this paragraph.

In some embodiments, the nucleic acid sequence/gene is placed under the control of an inducible promoter, which is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the proteins used in the present invention. For E. coli—and other bacterial host cells, inducible promoters are known to those of skill in the art.

Throughout the invention, the expression “gene” is meant to represent a linear sequence of nucleotides (or a nucleic acid sequence; see above) along a segment of DNA that provides the coded instructions for synthesis of RNA, which, when translated into protein, leads to the expression of a protein/peptide. The protein/peptide may—as in the present invention—have certain enzymatic functions. A “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof.

The term “nucleic acid sequence encoding . . . ” or “gene(s) encoding/coding for . . . ” generally refers to any polyribonucleotide or polydeoxyribonucleotide, which may be unmodified RNA or DNA or modified RNA or DNA, and generally represents a gene which encodes a certain polypeptide or protein. The term includes, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

Accordingly, in the present invention, the terms “gene” and “nucleic acid sequence” are used interchangeably.

Further, as used herein term “activity” when referring to an enzyme is meant to comprise any molecule displaying enzymatic activity, in particular a protein, and acting as a catalyst to bring about a specific biochemical reaction while remaining unchanged by the reaction. In particular, proteins with enzymatic activities are meant to be comprised by this term, which are able to convert a substrate into a product.

In enzymatic reactions, the molecules at the beginning of the process, called substrates, are converted into different molecules, called products. Almost all chemical reactions in a biological cell need enzymes in order to occur at rates sufficient for life. Since enzymes are selective for their substrates and speed up only a few reactions from among many possibilities, the set of enzymes made in a cell determines which metabolic pathways occur in that cell.

Accordingly, when, according to the invention, the activity of an enzyme is “abolished” or “lowered” the enzyme does not have the activity it has if the enzyme or its expression is unmodified, i.e. the activity, in that case, is abolished or lowered compared to the unmodified enzyme/enzyme expression.

The inventors of the present invention were able to provide for a method and a genetically modified host cell, by means of which fucosylated oligosaccharides could be produced with a product titer exceeding 100 g/L.

According to one embodiment of the invention of the method or the host cell of the invention, the activity of a fructose-6-phosphate-converting enzyme, which in the unmodified host cell is—where applicable—at a regular level, is lowered or abolished. When using E. coli as a host cell, it is preferred in the method and the host cell according to the invention the fructose-6-phosphate converting enzyme is selected from the group consisting of phosphofructokinase, preferably phosphofructokinase A (PfKA), glucose-6-phosphate isomerase, fructose-6-phosphate aldolase, a transketolase, preferably tktA, tktB, or a transaldolase, preferably talA, talB, which fructose-6-phosphate converting enzyme, being otherwise present and active in the unmodified E. coli host cell, has been modified such that its activity is lowered or abolished.

PfkA is efficiently phosphorylating fructose-6-phophate to fructose-1,6-bisphosphate. Fructose-6-phosphate is the branching point in glycolytic, and gluconeogenic pathways, and in the synthesis of GDP-L-fucose that starts from the ManA catalyzed isomerization of fructose-6-phosphate to mannose-6-phosphate. When growing E. coli on a gluconeogenic substrate like glycerol, the phosphorylation of fructose-6-phosphate by PfkA is a highly ATP consuming treadmill reaction and, in addition, it competes with ManA for the substrate.

According to an embodiment of the method and the host cell of the invention, the fructose-6-phosphate generating enzyme is fructose-1,6-bisphosphate phosphatase, the activity of which can be increased to increase the pool of fructose-6-phosphate.

According to another embodiment of the invention, at least one gene encoding an enzyme necessary for the de novo synthesis of GDP-fucose is overexpressed in the host cell.

GDP-fucose, as mentioned above, serves as L-fucose-donor for the reaction of the fucosyltransferase transferring L-fucose to an acceptor substrate to form the fucosylated oligosaccharide.

With the prokaryotic host cell genetically modified to internally produce GDP-fucose, an external addition of L-fucose, which can be converted to GDP-fucose via the salvage pathway, is not needed, since the host cell effectively produces GDP-fucose needed for the fucosylation-process of the desired oligosaccharide.

The at least one gene one encoding an enzyme necessary for the de novo synthesis of GDP-fucose and being overexpressed in the host cell can be an endogenous gene or an exogenous gene, which can be integrated into the genome of the host cell.

In an embodiment of the present invention, the exogenous genes encoding the enzymes necessary for the de novo synthesis of GDP-fucose are a gene coding for a phosphomannomutase, preferably manB, a gene coding for amannose-1-phosphate guanosyltransferase, preferably manC, a gene coding for a GDP-mannose-4,6-dehydratase, preferably gmd, and a gene coding for a GDP-L-fucose synthase, preferably wcaG.

According to an embodiment of the invention, and as mentioned further above, at least one exogenous gene encoding a fucosyltransferase is selected from a gene expressing a protein that exhibits an alpha-1,2-fucosyltransferase activity and/or an alpha-1,3-fucosyltransferase activity. In this connection it is particularly preferred if the alpha-1,2-fucosyltransferase is selected from the group consisting of wbgL of E. coli:O126 or the alpha-1,2-fucosyltransferase encoded by the fucT2 gene of Helicobacter pylori, and if the gene encoding an alpha-1,3-fucosyltransferase is selected from the group consisting of alpha-1,3-fucosyltransferases of the species Akkermansia muciniphila and Bacteroides fragilis, Helicobacter pylori or Helicobacter hepaticus.

According to another embodiment of the invention it is preferred if the host cell is further genetically modified: (i) to express an gene, preferably an exogenous gene, encoding a protein enabling or facilitating the export of the desired fucosylated oligosaccharide into the culture medium; and/or (ii) to express an exogenous gene encoding a bifunctional L-fucokinase/L-fucose 1-phosphate guanylyltransferase; and/or (iii) to exhibit mutated or deleted genes fucl and fucK leading to lowered or abolished activities of the L-fucose-isomerase (Fucl) and L-fuculose-kinase (FucK), (iv) and/or to have inactivated or disrupted genes encoding enzymes of the colonic acid synthesis; and/or (v) to express, preferably overexpress, an endogenous and/or exogenous permease for the import of lactose; and/or (vi) to have inactivated or deleted endogenous beta-galactosidase genes; and/or (vii) to express a gene encoding beta-galactosidase, preferably an exogenous regulable beta-galactosidase; and/or (viii) to overexpress an endogenous and/or exogenous gene coding for a fructose-1,6-bisphosphate phosphatase.

With the additional genetic modification as indicated above, the method for producing a fucosylated oligosaccharide could be even more improved.

With the deletion or inactivation of the genes encoding L-fucose isomerase (e.g., Fucl) and L-fuculose-kinase (e.g., FucK), the catabolism of intracellular fucose can be avoided.

With the disruption, deletion or inactivation of genes encoding enzymes of the colonic acid biosynthesis (e.g., in E. coli as host cell, the wcaJ gene that catalyzes the first step of colanic acid synthesis), the intracellularly production of colanic acid, which otherwise might compete with the fucosyltransferase reaction for the substrate GDP-L-fucose, is prevented.

According to a preferred embodiment, the protein that enables or facilitates the export of the desired fucosylated oligosaccharide into the culture medium is a sugar efflux transporter preferably selected from yberc0001_9420 and E. coli SetA.

According to a preferred embodiment, the gene encoding a bifunctional L-fucokinase/L-fucose 1-phosphate guanylyltransferase is fkp from Bacteroides fragilis.

According to a preferred embodiment, the fructose-1,6-bisphosphate phosphatase is encoded by a gene which is a functional active variant of the fructose-1,6-bisphosphate phosphatase (fbpase) from Pisum sativum.

According to a preferred embodiment, the lactose permease is E. coli LacY.

With the expression of a gene encoding a bifunctional L-fucokinase/L-fucose 1-phosphate guanylyltransferase that catalyzes the synthesis of GDP-L-fucose, e.g. of the Bacteroides fragilis fkp gene, the formation of free L-fucose that could accumulate as a byproduct following the hydrolysis of GDP-L-fucose is prevented, thereby rescuing free L-fucose for the synthesis of the desired fucosylated oligosaccharide.

According to a preferred embodiment, the exogenous genes for metabolizing galactose are the genes comprising the galETKM operon and/or galP from E. coli.

According to an embodiment of the invention, the genes the host cell is modified in or with are endogenous or exogenous genes.

Throughout the invention, and applying for each gene/nucleic acid that has been exogenously introduced into the host cell, it is—according to an embodiment of the present method and host cell—preferred, if at least one of the exogenous genes, preferably integrated in the host cell genome, is overexpressed, preferably upon endogenous or exogenous induction, or in a constitutive manner.

Accordingly, it is preferred if at least one of the following gene(s) is/are overexpressed: (i) exogenous genes encoding enzymes necessary for the de novo synthesis of GDP-fucose; (ii) an exogenous gene encoding a fucosyltransferase; (iii) an exogenous gene encoding a sugar efflux transporter; (iv) an exogenous gene encoding a bifunctional L-fucokinase/L-fucose 1-phosphate guanylyltransferase; (v) a lactose permease; (vi) an exogenous regulable gene encoding a beta-galactosidase; and/or (vii) exogenous genes for metabolizing galactose; (viii) an exogeneous gene encoding a fructose-1,6-bisphospahate phosphatase; the overexpression can be effected, e.g., by means of a regulable promoter that initiates transcription of the gene(s), either at a certain time point or period during the cultivation or for the whole cultivation time.

Also, according to a preferred embodiment, the exogenous genes to be introduced in the host cell employed in the method according to the invention are integrated into the genome of the host cell.

Also, according to one aspect of the invention, and unless otherwise defined, the genes the host cell is modified in/with according to the invention can also be endogenous genes, and their expression can be enhanced or increased or overexpressed, or otherwise abolished or decreased.

With the inactivation or deletion of endogenous beta-galactosidase gene(s) the degradation of externally added lactose is prevented; however, since it is desirable to have lactose degraded that is not metabolized and that would otherwise impede the purification of the desired fucosylated oligosaccharide, it is also preferred if an exogenous regulable gene encoding a beta-galactosidase or a mutated form of the beta-galactosidase is expressed in the host cell. E.g., the lacZΩ fragment of the lacZ gene can be expressed, the expression of which, e.g., can be regulated by means of a repressor, e.g. by a temperature sensitive transcriptional repressor, e.g. cl857. In this case, synthesis of the beta-galactosidase Ω-fragment can be initiated by raising the temperature to 42° C.

A repressor, as presently and generally in the state of the art understood, is a DNA- or RNA-binding protein that inhibits the expression of one or more genes by binding to the operator or associated silencers. A DNA-binding repressor blocks the attachment of RNA polymerase to the promoter, thus preventing transcription of the genes into messenger RNA.

As a promoter for the exogenous beta-galactosidase alpha fragment, e.g., the E. coli BL21 (DE3) PgbA promoter can be used. The beta-galactosidase α- and Ω-fragments are combined to result in an active beta-galactosidase in the cell.

In a preferred embodiment, the lactose is provided by adding lactose from the beginning of the cultivation in a concentration of at least 5 mM, more preferably in a concentration of more than 10, 20, 30, 40, 50, 60, 70, 80, 90 or 100 mM, or even more in a concentration of 300 mM or higher than 300, or 400 mM.

According to yet another embodiment, lactose is provided by adding lactose the cultivation medium in a concentration, such, that throughout the production phase of the cultivation a lactose concentration of at least 5 mM, more preferably of at least 10, 20 or 30 mM, is obtained.

Alternatively, lactose can be produced by the host cell intracellularly, as described in patent EP1927316 A1, the content of which is herewith expressively referred to and incorporated by reference.

In the method according to the invention, it is preferred if the host cells are cultivated for at least about 60, 80, 100 or about 120 hours or in a continuous manner.

Thus, according to one aspect of the invention, i.e. in a continuous method, a carbon source is constantly added to the medium during the cultivating step of the host cell. By constantly adding the carbon source during the cultivation step, a constant and effective production of the oligosaccharide is accomplished.

According to another aspect, the method according to the invention is or includes a fed-batch fermentation method, with either a constant volume fed-batch culture, where the substrate is fed without diluting the culture, or variable volume fed-batch culture, where the fermentation volume changes with the fermentation time due to the substrate feed.

As mentioned above, the present invention also concerns a genetically modified prokaryotic host cell, which host cell has been genetically modified such, that (i) the activity of a fructose-6-phosphate-converting enzyme, which in the unmodified host cell is at a regular level, is lowered or abolished; (ii) at least one gene encoding an enzyme necessary for the de novo synthesis of GDP-fucose is overexpressed; (iii) an exogenous gene encoding a fucosyltransferase, preferably a gene encoding an alpha-1,2-fucosyltransferase and/or an alpha-1,3-fucosyltranferase, is expressed in the cell.

As mentioned for the method above, the host cell is preferably selected from a Escherichia coli strain, a Lactobacillus strain or a Corynebacterium strain.

According to an embodiment of the host cell according to the invention, the intracellular pool of fructose-6-phosphate is increased by (i) lowering or abolishing the activity of a fructose-6-phosphate converting enzyme that is selected from the group of phosphofructokinase, glucose-6-phosphate isomerase, fructose-6-phosphate aldolase, a transketolase, e.g. tktA, tktB, or a transaldolase, e.g. talA, talB, or (ii) increasing the fructose-1,6-bisphosphate phosphatase activity.

In a preferred embodiment, the genes encoding enzymes necessary for the de novo synthesis of GDP-fucose are overexpressed.

In yet another preferred embodiment, the exogenous genes encoding at least one fucosyltransferase are genes encoding alpha-1,2-fucosylltransferases and/or alpha-1,3-fucosyltransferases and are selected from wbgL from E. coli O126 or fucT2 from Helicobacter pylori, referring to alpha-1,2-fucosylltransferases, and genes of the species Akkermansia muciniphila, Bacteroides fragilis, Helicobacter pylori, or Helicobacter hepaticus, referring to alpha-1,3-fucosyltransferases.

According to an embodiment, the host cell as described above is optionally genetically further modified (iv) to express an exogenous gene encoding a sugar efflux transporter; and/or (v) to express an exogenous gene encoding a bifunctional L-fucokinase/L-fucose 1-phosphate guanylyltransferase; and/or (vi) to have inactivated or deleted genes encoding a L-fucose-isomerase and L-fuculose-kinase; and/or (vii) to have inactivated or disrupted genes encoding the UDP-glucose:undecaprenyl phosphate glucose-1-phosphate transferase; and/or (viii) to express an exogenous lactose permease; and/or (ix) to have inactivated or deleted endogenous beta-galactosidase genes; and/or (x) to express an exogenous regulable gene encoding a beta-galactosidase; and/or (xi) to express exogenous genes for metabolizing galactose; and/or to express an exogenous gene encoding a fructose-1,6-bisphosphare phosphatase.

The present invention also concerns the use of the genetically modified prokaryotic host cell according to the invention for the production of a fucosylated oligosaccharide

Further advantages follow from the description of the embodiments and the attached drawings.

It goes without saying that the abovementioned features and the features which are still to be explained below can be used not only in the respectively specified combinations, but also in other combinations or on their own, without departing from the scope of the present invention.

### BRIEF DESCRIPTION OF THE FIGURES

Several embodiments of the invention are illustrated in the figures and explained in more detail in the following description. In the figures:

FIG. 1 A schematic, exemplary illustration of a genetically modified host cell to be used in the method according to the invention;

FIG. 2 HPLC analyses of supernatants from glycerol grown cultures of 2′-fucosyllactose producing E. coli strains by HPLC; and

FIG. 3 SEQ ID NOs: 1-7.

### DETAILED DESCRIPTION OF THE FIGURES AND EMBODIMENTS

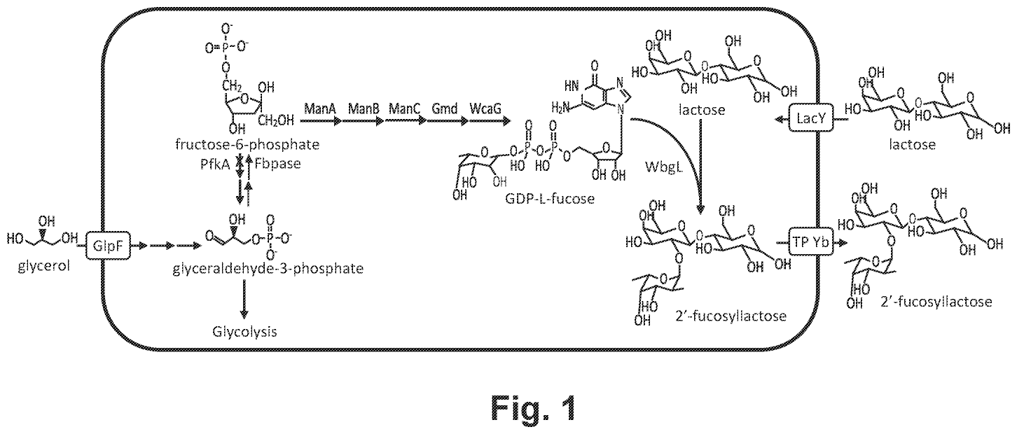

FIG. 1 shows an exemplary illustrative host cell according to the invention to be employed in the method according to the invention, with exemplary pathways for the fermentation of the exemplary fucosylated oligosaccharides 2′-fucosyllactose being depicted. In FIG. 1, an exemplary bacterial host cell is shown that has been genetically modified according to the invention, with respect to the production of 2′-fucosyllactose.

As can be seen from FIG. 1, glycerol is exemplary used as carbon source, while lactose is externally added. Lactose is transported into the host cell via a permease (e.g. LacY). Glycerol is taken up into the host cell via facilitated diffusion through GlpF. Within the prokaryotic host cell, glycerol is converted into glyceraldehyde-3-phosphate, which is converted to fructose-6-phosphate, (i) favored by the overexpression of an exogenous gene encoding a Fbpase and (ii) with the reverse reaction being inhibited by the inactivation of the phosphofructokinase A (PfkA). Via overexpression of exogenous enzymes necessary for the de novo synthesis of GDP-fucose, i.e. phosphomannomutase ManB, mannose-1-phosphate guanosyltransferase ManC, GDP-mannose-4,6-dehydratase Gmd, and GDP-L-fucose synthase WcaG, GDP-L-fucose is produced.

In a next step, GDP-L-fucose, by the action of an alpha-1,2-fucosyltransferase, e.g. WbgL, reacts with the internalized lactose to produce 2-fucosyllactose, which is exported via an efflux transporter, e.g. TPYb, into the medium the host cell is cultivated in.

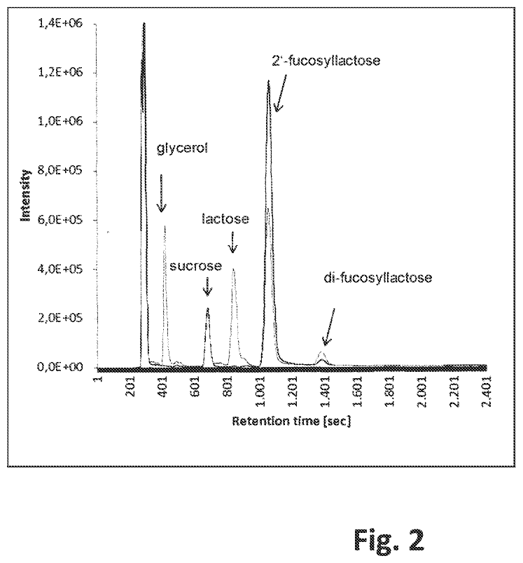

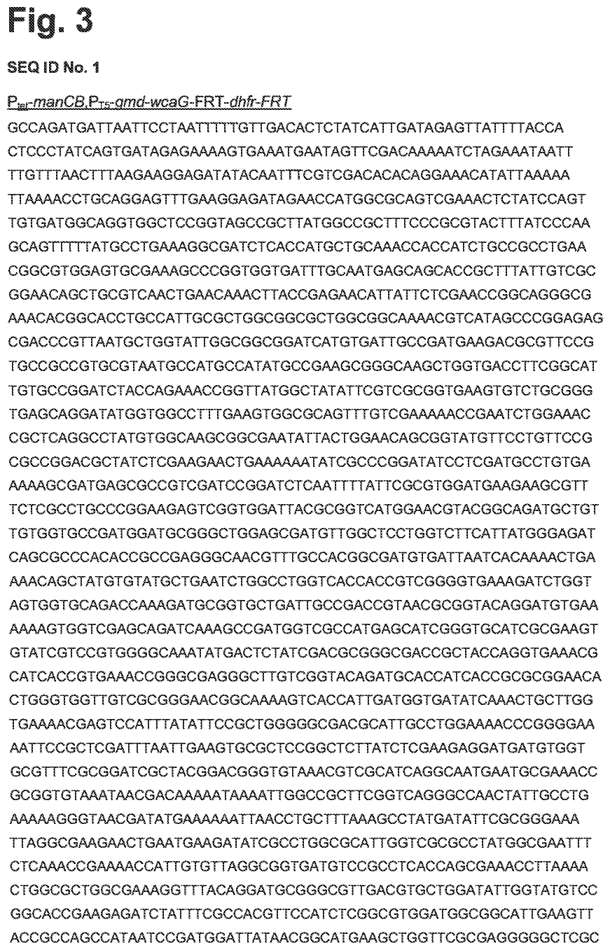

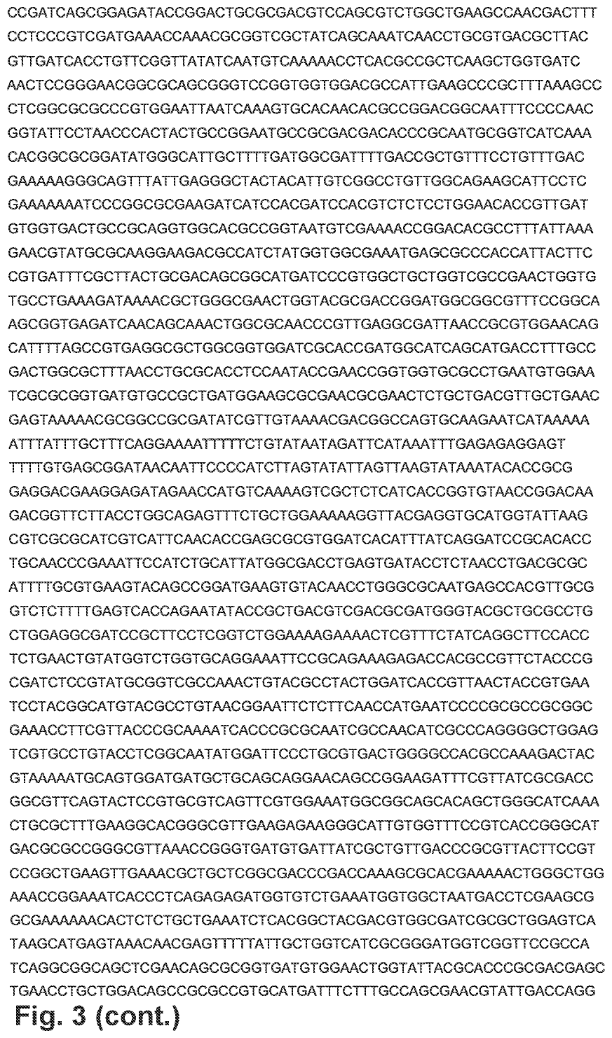

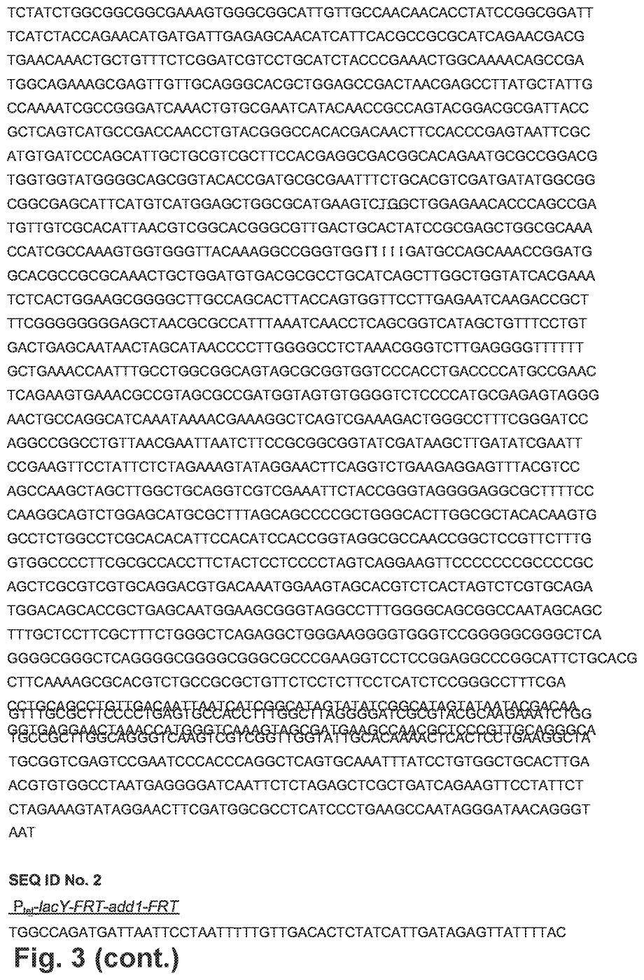

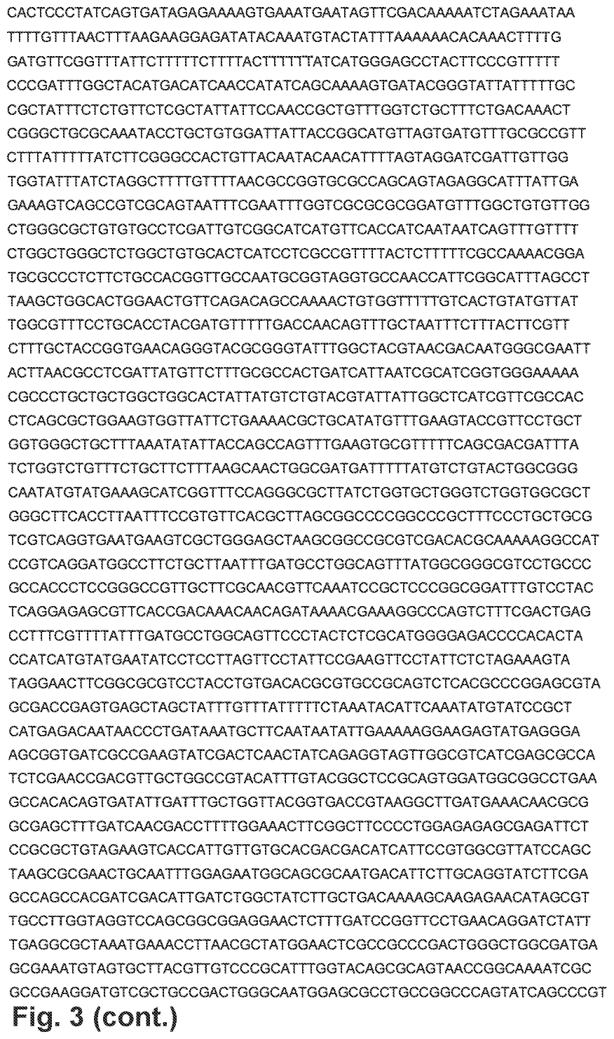

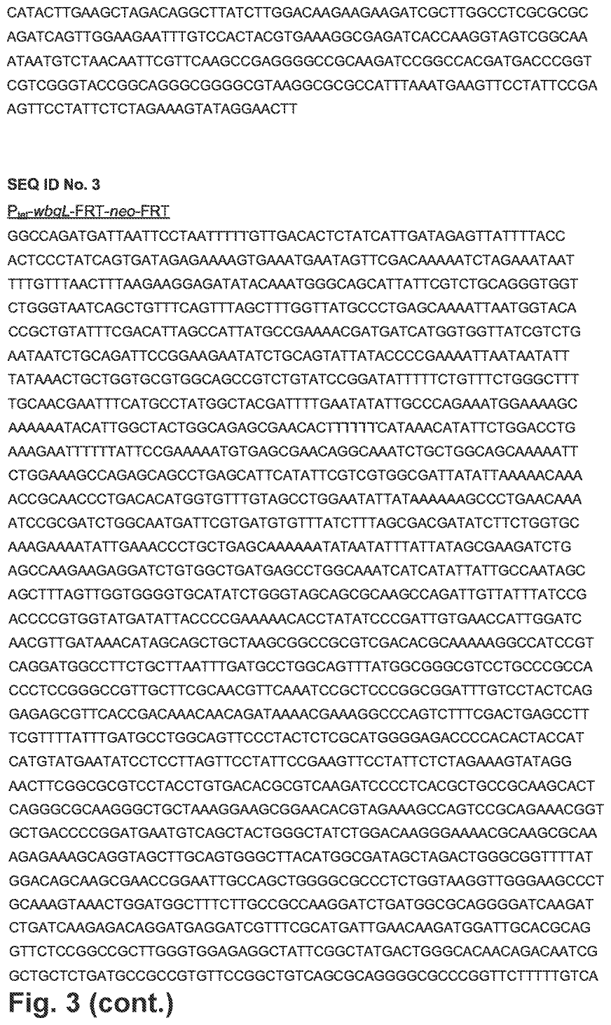

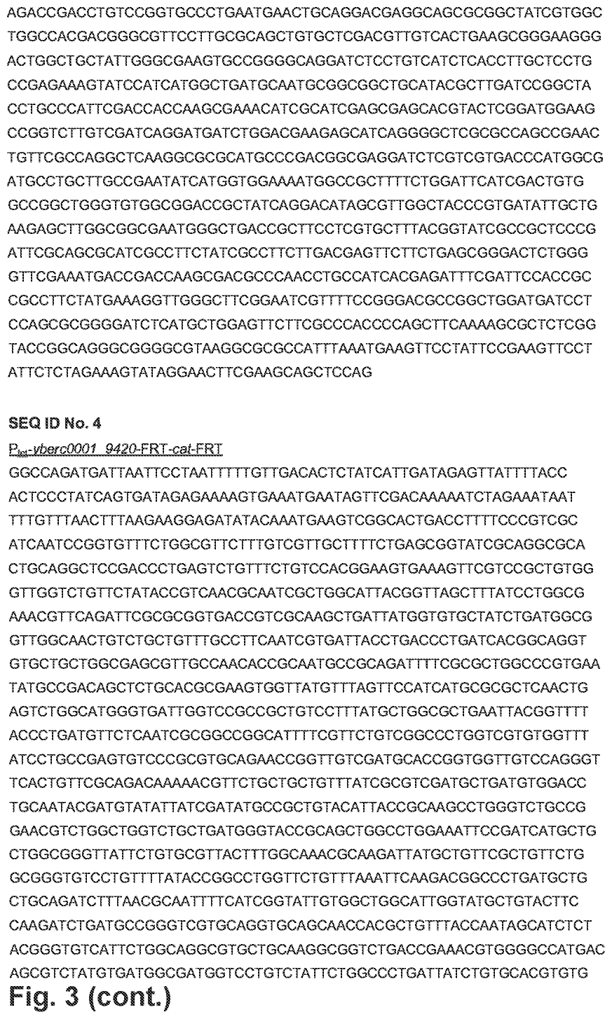

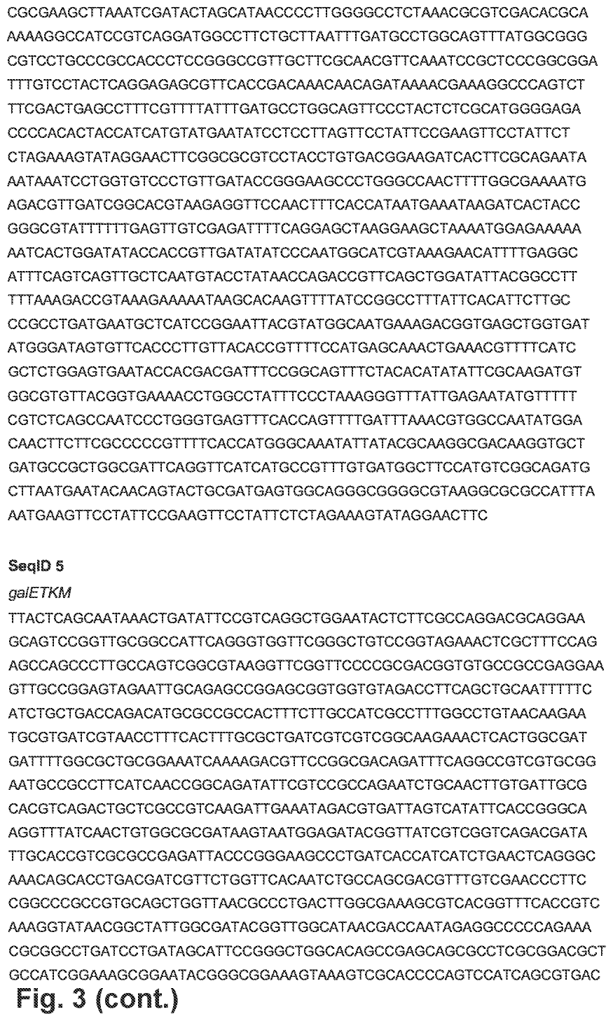

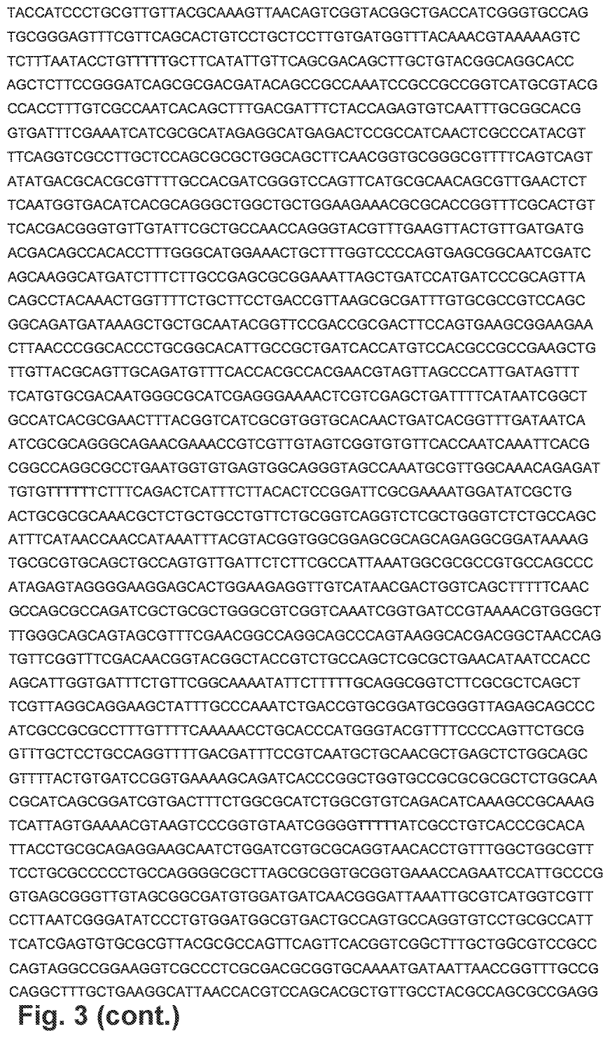

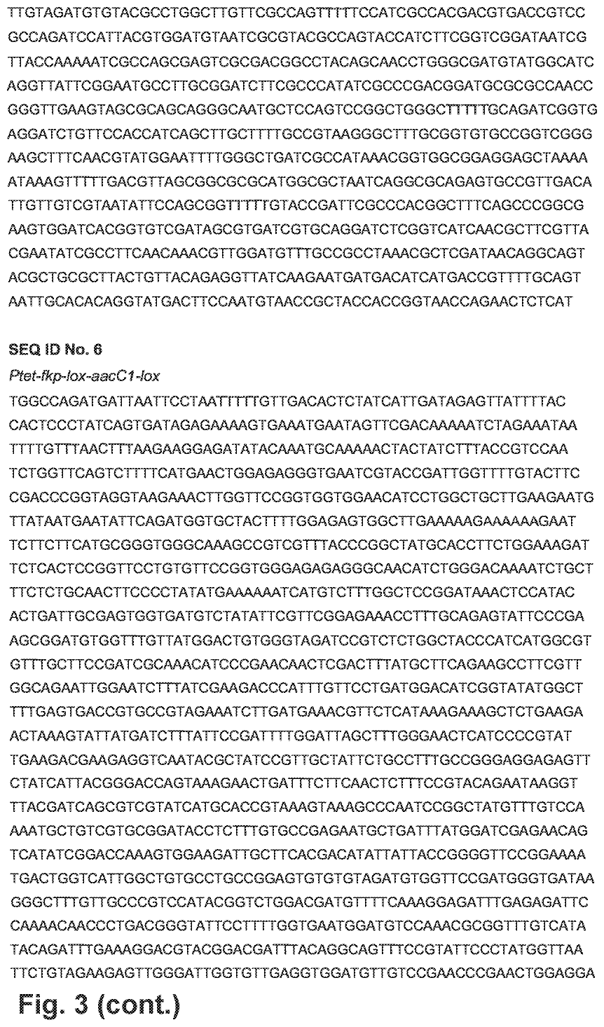

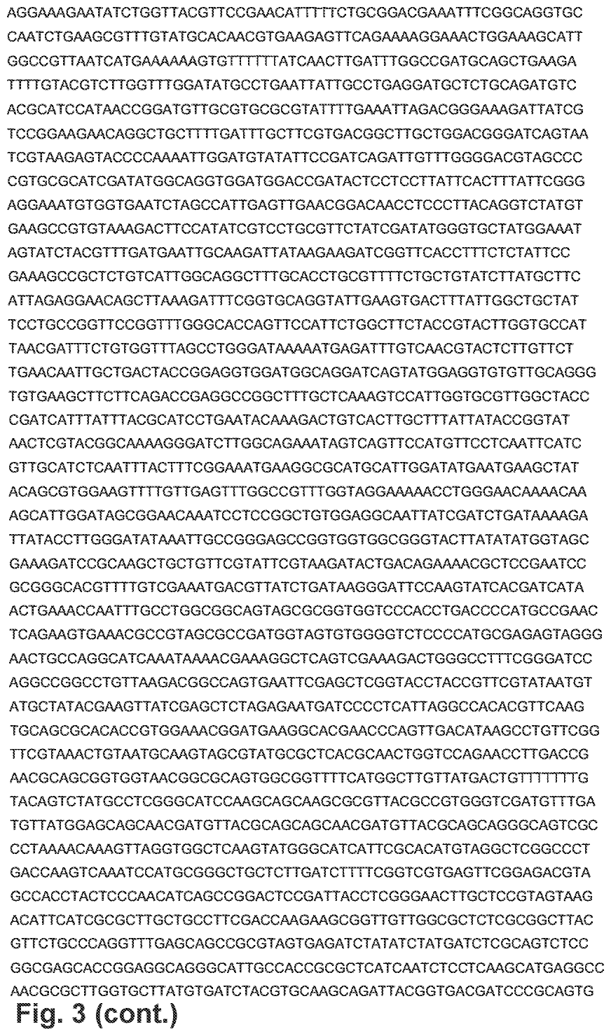

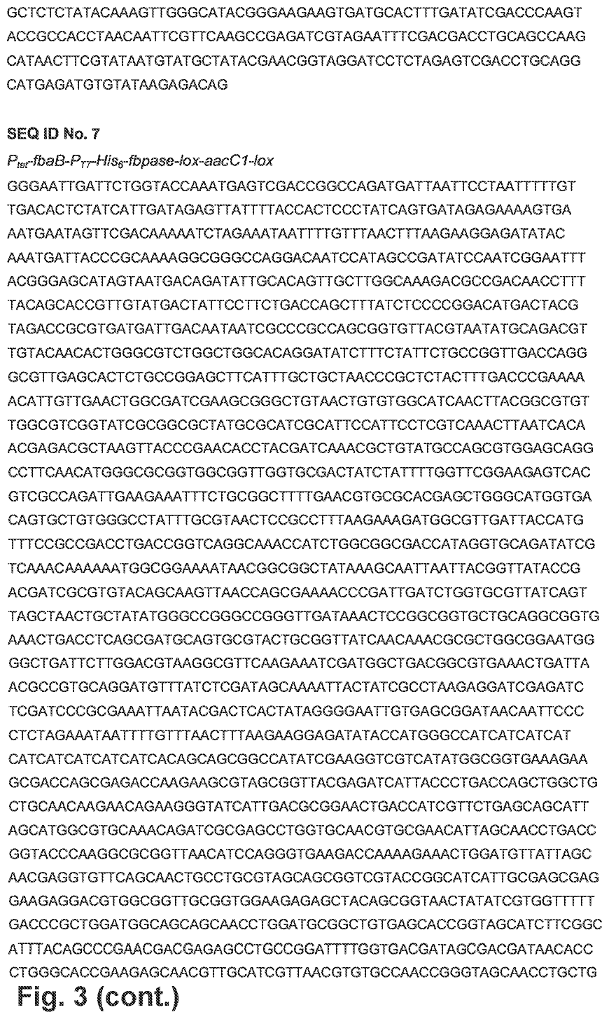

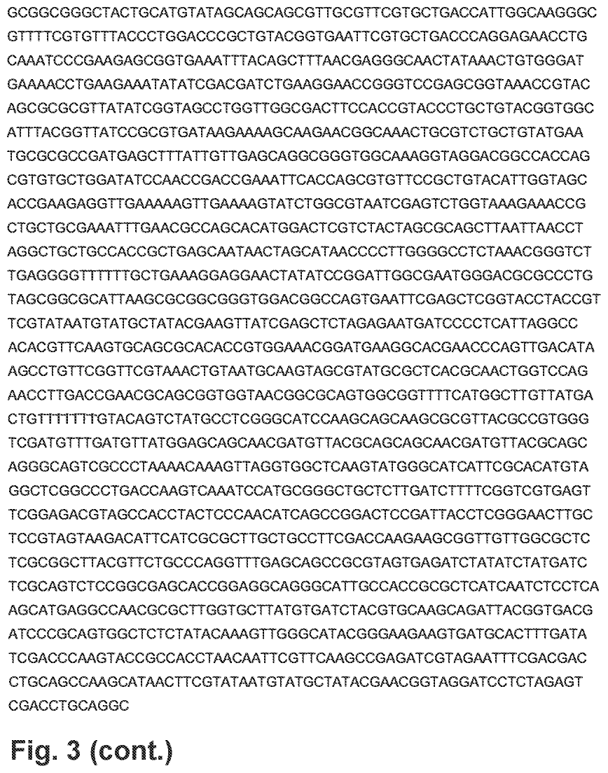

In FIG. 2 the results of HPLC analyses of supernatants from glycerol grown cultures of 2′-fucosyllactose producing E. coli strains by HPLC are shown.

Depicted in FIG. 2 is the HPLC profile of the fermentation broth from a 2′-fucosyllactose producing strain harbouring the gene encoding the heterologous transporter yberc0001_9420 (black) and the HPLC profile of fermentation broth of the same strain after deletion of the gene encoding the heterologous transporter yberc0001_9420 (gray). Fermentation of both strains was conducted for 111 h at 28° C., using glycerol as source of carbon and energy.

### Example 1

Engineering of an E. coli BL21(DE3) Strain for the Production of 2′-Fucosyllactose

Using E. coli BL21(DE3) as parental host a strain for the production of 2′-fucosyllactose in a whole cell biosynthetic approach was constructed. Genomic engineering of the strain included gene disruption and deletion events and integration of heterologous genes.

Since 2′-fucosyllactose is synthesized from lactose, that is applied to the bacterial culture, and from GDP-L-fucose that is produced from the living cells, first the wild-type copy of the lacZ gene encoding the endogenous β-galactosidase was inactivated by mutagenesis using mismatch oligonucleotides (see Ellis et al., “High efficiency mutagenesis, repair, and engineering of chromosomal DNA using single-stranded oligonucleotides”, Proc. Natl. Acad. Sci. USA 98: 6742-6746 (2001). Using the same method, the gene for the arabinose-isomerase araA was disrupted.

A lacZΩ gene fragment was introduced under the control of the temperature sensitive transcriptional repressor cl857. The lacZα fragment gene is expressed under the control of the E. coli BL21 (DE3) PgbA promoter in the strain, revealing a LacZ⁺ strain.

Genomic deletions were performed by λ Red mediated recombination according to the method of Datsenko and Warner (see “One-step inactivation of chromosomal genes in Escherichia coli K-12 using PCR products”, Proc. Natl. Acad. Sci. USA 97:6640-6645 (2000)). The genes fucl and fucK, coding for the L-fucose isomerase and the L-fuculose kinase, respectively, have been deleted to prevent degradation of L-fucose. Also genes wzxC-wcaJ were deleted. WcaJ probably encodes a UDP-glucose:undecaprenyl phosphate glucose-1-phosphate transferase catalysing the first step in colanic acid synthesis (see Stevenson et al., “Organization of the Escherichia coli K-12 gene cluster responsible for production of the extracellular polysaccharide colonic acid”, J. Bacteriol. 178:4885-4893; (1996)); production of colanic acid would compete for GDP-fucose with the fucosyltransferase reaction.

Genomic integration of heterologous genes was performed by transposition. Large gene clusters were integrated into the genome mediated by the hyperactive C9-mutant of the mariner transposase Himar1 (see Lampe et al., “Hyperactive transposase mutants of the Himar1 mariner transposon”, Proc. Natl. Acad. Sci. USA 96:11428-11433 (1999)), that was inserted into the plasmid pEcomar under transcriptional control of the Pₐᵣₐ promotor. To enhance de novo synthesis of GDP-fucose, genes encoding phosphomannomutase (manB), mannose-1-phosphate guanosyltransferase (manC), GDP-mannose-4,6-dehydratase (gmd), and GDP-L-fucose synthase (wcaG) from E. coli K12 DH5α were overexpressed in the E. coli BL21(DE3) strain; the operon manCB was set under control of the constitutive promoter Pₜₑₜ, the operon gmd, wcaG is transcribed from the constitutive P_{T5} promoter. The transposon cassette <Pₜₑₜ-manCB-P_{T5}-gmd, wcaG-FRT-dhfr-FRT>(SEQ ID No. 1), including the gene for the dihydrofolate reductase for trime-thoprim resistance, flanked by the inverted terminal repeats specifically recognized by the mariner-like element Himar1 transposase was inserted into the E. coli genome from pEcomar C9-manCB-gmd, wcaG-dhfr.

For chromosomal integration of single genes, the EZ-Tn5™ transposase (Epicentre, USA) was used. To produce EZ-Tn5 transposomes the gene of interest together with a FRT-site flanked antibiotic resistance cassette was amplified with primers that carried on both sites the 19-bp Mosaic End recognition sites (5′-CTGTCTCTTATAC ACATCT (SEQ ID No. 8)) for the EZ-Tn5 transposase. Using the EZ-Tn5™ transposase, the gene for the lactose importer LacY from E. coli K12 TG1 (acc. no. ABN72583), the 2-fucosyltransferase gene wbgL from E. coli:O126 (acc. no. ADN43847), and the gene yberc0001_9420 encoding a sugar efflux transporter of the major facilitator superfamily from Yersinia bercovieri ATCC 43970 (acc. no. EEQ08298) were integrated using the respective integration cassettes: <Pₜₑₜ-lacY-FRT-aadA-FRT>(SEQ ID No. 2), <Pₜₑₜ-wbgLco-FRT-neo-FRT>(SEQ ID No. 3), and <Pₜₑₜ-yberc0001_9420co-FRT-cat-FRT>(SEQ ID No. 4), yielding strain. The genes wbgL and yberc0001_9420 were synthetically synthesized and codon optimized (co) by GenScript Cooperation (USA). After successful integration of the lacY gene the resistance gene was eliminated from streptomycin resistant clones by the FLP recombinase encoded on plasmid pCP20 (Datsenko and Warner, “One-step inactivation of chromosomal genes in Escherichia coli K-12 using PCR products”, Proc. Natl. Acad. Sci. USA 97:6640-6645 (2000)).

Since E. coli BL21(DE3) lacks a functional gal-operon a natively regulated copy of the galETKM operon from E. coli K was integrated into the B strain by EZ-transposition using integration cassette <P_{gal}-galE-galT-galK-gaIM>(SEQ ID No. 5). Integrands were selected from MacConkey-agar containing 1% galactose as red colonies. The resulting strain is able to metabolize the monosaccharides glucose and galactose originating from lactose hydrolysis.

### Example 2

Verification of Enhanced 2′-Fucosyllactose Export by Yersinia bercovieri ATCC 43970 Sugar Efflux Transporter
Knock-Out of yberc0001 9420

To demonstrate functionality of the heterologous sugar transporter from Yersinia bercovieri ATCC 43970 the gene yberc0001_9420 was deleted from strain E. coli BL21(DE3) lacZ, araA, fucl , fucK, wcaJ, that contained chromosomal integrations of manB, manC, gmd, wcaG, lacY, wbgL; and yberc0001_9420 by homologous recombination according to Datsenko and Wanner (2000; see above) using the gentamycine resistance cassette aacC1 from plasmid pBBR-MCS5 (Kovach, Elzer et al. 1995, “Four new derivatives of the broad-host-range cloning vector pBBR1MCS, carrying different antibiotic-resistance cassettes”, Gene 166, 175-176), that was inserted into gene yberc0001_9420, yielding strain Δyberc0001_9420.

### Cultivation Conditions for 2′-Fucosyllactose Production

The E. coli BL21 (DE3) strain harbouring the heterologous exporter yberc0001_9420 and the Δyberc0001_9420 strain were cultivated at 28° C. in 3 L fermenters (New Brunswick, Edison, USA) starting with 800 mL mineral salts medium containing 7 g/L NH₄H₂PO₄, 7 g/L K₂HPO₄, 2 g/L KOH, 0.3 g/L citric acid, 2 g/L MgSO₄×7H₂O, and 0.015 g/L CaCl₂×6H₂O, supplemented with 1 mL/L trace element solution (54.4 g/L ammonium ferric citrate, 9.8 g/L MnCl₂×4H₂O, 1.6 g/L CoCl₂×6H₂O, 1 g/L CuCl₂×2H₂O, 1.9 g/L H₃BO₃, 9 g/L ZnSO₄×7H₂O, 1.1 g/L Na₂MoO₄×2H₂O, 1.5 g/L Na₂SeO₃, 1.5 g/L NiSO₄×6H₂O) containing 1.5% glycerol as carbon source and the antibiotics trimethoprim 10 μg/ml, and kanamycin 15 μg/ml. Cultivation was started with a 2.5% (v/v) inoculum from a pre-culture grown in the same glycerol containing medium. Lactose as acceptor in the fucosyltransferase reaction was added within seven hours to obtain a concentration of 30 mM in the culture, starting at OD660ₙₘ of about 10. Lactose was then adjusted manually to maintain an excess of the acceptor molecule; glycerol was added continuously.

### Analysis of Culture Supernatant and Detection of 2′-Fucosyllactose by HPLC

Analysis by high performance liquid chromatography (HPLC) was performed using a refractive index detector (RID-10A) (Shimadzu, Germany) and a ReproSil Carbohydrate, 5 μm (250 mm×4,6 mm) (Dr. Maisch GmbH, Germany) connected to an HPLC system (Shimadzu, Germany). Elution was performed isocratically with acetonitril:H₂O (68/32 (v/v)) as eluent at 35° C. and a flow rate of 1.4 ml/min. 20 μl of the sample were applied to the column. 2′-fucosyllactoseconcentration was calculated from a standard curve. Therefore, 10% (v/v) 100 mM sucrose were added to the HPLC samples as internal standard before they were filtered (0,22 μm pore size) and cleared by solid phase extraction on an ion exchange matrix (Strata ABW, Phenomenex).

Detection of 2′-Fucosyllactose in Supernatants of E. coli BL21(DE3) Cultures

After 111 h of fermentation at 28° C. in mineral salts medium with glycerol as carbon source, 73 mM (35,6 g/L) and 25 mM (12,2 g/L) 2′-fucosyllactose were detected by HPLC in the culture supernatant of strains containing, and lacking the yberc0001_9420 transporter gene, see FIG. 2: Depicted in FIG. 2 is the HPLC profile of the fermentation broth from a 2′-fucosyllactose producing strain harbouring the gene encoding the heterologous transporter yberc0001_9420 (black) and the HPLC profile of fermentation broth of the same strain after deletion of the gene encoding the heterologous transporter yberc0001_9420 (gray). Deletion of the heterologous sugar exporter yberc0001_9420 in the strain decreases the detected amount of 2′-fucosyllactose in the supernatant. That gives evidence, that indeed the transporter protein enhances 2′-fucosyllactose production by faster transport of the tri-saccharide outside the cell, since the genetic background despite the yberc0001_9420 gene is identical in both strains. Additionally, a lower cell density was achieved in the cells lacking the 2′-fucosyllactose exporter, probably due to osmotic stress caused by strong sugar accumulation inside the cells. As shown in FIG. 2, the amount of 2′,3-difucosyllactose detected in the Δyberc0001_9420 culture is about double than in the broth of the original strain. Increased production of 2′,3-difucosyllactose, where L-fucose is transferred to 2′-fucosyllactose by a fucosyltransferase catalyzed reaction, also suggests higher intracellular concentrations of the acceptor molecule 2′-fucosyllactose in the yberc0001_9420 knock-out strain as compared to the yberc0001_9420 overexpression strain.

In FIG. 2, the lighter lines, i.e. the grey lines, display the supernatant of the Δyberc0001_9420 E. coli BL21(DE3) strain Δyberc0001_9420 , the black lines display the supernatant of the culture of the yberc0001_9420 containing E. coli BL21(DE3). Samples were taken after 111 h of fermentation at 28° C. in mineral salts medium using glycerol as carbon source.

### Example 3

Production of 2′-Fucosyllactose in a Fermentative Process

Fermentations were conducted in 3 L-fermenters at 30° C. and at pH 7.0; the pH was regulated by titration with 25% ammonia. The strain described in example 2 was cultivated in the mineral salts medium described in example 2 using glycerol as source of carbon and energy. The fermenter with a starting volume of 1 L was inoculated with a pre-culture cultivated in the same medium. After consumption of the 2% glycerol contained within the batch, glycerol (60% v/v) was fed continuously. Lactose in a concentration of 0.66 M was added in three portions (in an one hour interval) of 10 mL each when an OD₆₀₀ₙₘ of 6 was reached. Afterwards, lactose was given in a continuous flow to hold a lactose concentration of at least 10 mM in the fermenter. After 86 h of cultivation a final titer of 91,3 mM (44,6 g/L) 2′-fucosyllactose was reached. By shifting the temperature to 42° C., the β-galactosidase gene is expressed and lactose and its degradation products glucose and galactose are metabolized by the 2′-fucosyllactose production strain.

### Example 4

HPLC-Analysis of Culture Supernatant

Analysis by HPLC was performed using a refractive index detector (RID-10A) (Shimadzu, Germany) and a Waters XBridge Amide Column 3.5 μm (250×4.6mm) (Eschborn, Germany) connected to an HPLC system (Shimadzu, Germany). Elution was performed isocratically with 30% A: 50% (v/v) ACN in ddH₂O, 0.1% (v/v) NH₄OH and 70% B: 80% (v/v) ACN in ddH₂O, 0.1% (v/v) NH₄OH (v/v) as eluent at 35° C. and at a flow rate of 1.4 ml/min. 10 μl of the sample were applied to the column, and the 2′-fucosyllactose concentration was calculated from a standard curve. Therefore, 10% (v/v) of a 100 mM sucrose solution was added to the HPLC samples as an internal standard prior to filtering (0.22 μm pore size) and clearing by solid phase extraction on an ion exchange matrix (Strata® ABW, Phenomenex). By-products like L-fucose, 3-fucosyllactose, 2′,3-difucosyllactose, and fucosylgalactose were also detected using the same analysis conditions.

### Example 5

### Improvement of 2′-Fucosyllactose Production Strain by Metabolic Engineering

Further improvement concerning the synthesis of 2′-fucosyllactose by the E. coli strain was achieved by deletion of the pfkA gene, encoding the phosphofructo-kinase A. When cultivating E. coli on a gluconeogenic substrate like glycerol the phosphorylation of fructose-6-phosphate by PfkA is a highly ATP consuming treadmill reaction and, in addition, it competes with ManA for the substrate. The pfkA gene was deleted by homologous recombination according to Datsenko and Wanner (2000, see above) using a gentamycin resistance cassette (aacC1) that was flanked by lox71/66 sites (see Lambert, Bongers et al. 2007 “Cre-lox-based system for multiple gene deletions and selectable-marker removal in Lactobacillus plantarum”, Appl. Environ. Microbial. 73, 1126-113). After successful deletion the pfkA gene the antibiotic resistance gene was removed from E. coli genome using the Cre recombinase (see Abremski, Hoess et al. 1983, “Studies on the properties of P1 site-specific recombination: evidence for topologically unlinked products following recombination”, Cell 32, 1301-1311) that was cloned under the control of the Pₐᵣₐ promoter in the pKD46 (see Datsenko and Wanner, 2000) chassis.

For different fucosyltransferases besides the transferase activity a GDP-L-fucose hydrolase activity was demonstrated. Also for wbgL, the alpha-1,2-fucosyltransferase used here for 2′-fucosyllactose synthesis this hydrolytic activity was shown (see EP3050973 A1). To rescue free L-fucose for the 2′-fucosyllactose production and to eliminate the contaminating L-fucose from the culture broth, the fkp gene, encoding the bifunctional L-fucokinase/L-fucose 1-phosphat guanylyltranferase of Bacteroides fragilis, under transcriptional control of the Pₜₑₜ promoter, together with the lox71/66 flanked aacC1 gene was chromosomally integrated into the strain described in example 1 by transposition using the EZ-Tn5™ transposase, <Ptet-fkp-lox-aacC1-lox>(SEQ ID No. 6). After successful integration the gentamycin resistance gene was removed from the genome as described above.

### Example 6

### Optimized Fermentation Process for the Production of 2′-Fucosyllactose

Using an optimized mineral salts medium that contains 3 g/L KH₂PO₄, 12 g/L K₂HPO₄, 5 g/L (NH₄)₂SO₄, 0.3 g/L citric acid, 2 g/L MgSO₄×7H₂O, 0.1 g/L NaCl and 0.015 g/L CaCl₂×6H₂O with 1 mL/L trace element solution (54.4 g/L ammonium ferric citrate, 9.8 g/L MnCl₂×4H₂O, 1.6 g/L CoCl₂×6H₂), 1 g/L CuCl₂×2H2O, 1.9 g/L H₃BO₃, 9 g/L ZnSO₄×7H₂O, 1.1 g/L Na₂MoO₄×2H₂O, 1.5 g/L Na₂SeO₃, 1.5 g/L NiSO₄×6H₂O) and 2% glycerol as carbon source batch, the E. coli strain described in example 5 was cultivated in a 3 L fermenter at 33° C. The pH was hold at 7.0 by titrating 25% ammonia. The fermenter was inoculated to an OD₆₀₀ₙₘ of 0.1 with a pre-culture grown in the same medium. Lactose was added when the culture obtained an OD₆₀₀ₙₘ of 5, to obtain a concentration of 30 mM. A concentration of 20-30 mM lactose was held throughout the whole fermentation process, regulated according to HPLC-analyses. Glycerol feeding (60% v/v) started after the glycerol in the batch was consumed with flow rates of 4.5 ml/L/h for 20 hours, followed by feeding for 33 hours with 5.7 ml/L/h and 18 hours for 7.1 ml/L/h over a period of 18 hours (feeding rates are referring to the starting volume). Overall, after 93 h a 2′-fucosyllactose titer of 106.5 g/L (217 mM) was obtained.

### Example 7

### Engineering of an Enhanced 2′-Fucosyllactose Production Strain by Metabolic Challenging

To enhance the flux of the metabolized carbon source glycerol through the gluconeogentic pathway from triose-phosphates to fructose-6-phophate to feed the GDP-L-fucose biosynthesis the genes encoding the fructose-1,6-bisphosphate aldolase (fbaB) and a heterologous fructose-1,6-bisphosphate phosphatase (fbpase) from Pisum sativum were overexpressed in the strain described in example 5. The fbaB gene from E. coli BL21 (DE3) was fused with the Ptet promoter. The activity of the chloroplastic P. sativum FBPase is allosterically regulated by a disulfide-dithiol exchange due to reduction by thioredoxins. Exchange of the cysteine residue 153 to serine results in a constitutively active enzyme. The gene encoding the chloroplastic FBPase from P. sativum (acc. No. AAD10213) was purchased codon optimized for expression in E. coli, N-terminally tagged with a hexahistidine-tag and modified to encode the C153S variant of the enzyme from Genescript. The fbpase gene is transcribed from a T7 promoter. The cassette <Pₜₑₜ-fbaB-P_{T7}-His₆-fbpase-lox-aacC1-lox>(SEQ ID No. 7) was used for EZ-Tn5™ transposase mediated integration in the host strain. After removal of the gentamycin resistance gene from the E. coli genome the strain was used for 2′-fucosyllactose production.

### Example 8

### Production of 150 g/L 2′-Fucosyllactose by a Fermentation Process

The 2′-fucosyllactose production strain genetically modified das described in example 7 was cultivated in the same medium at 33° C. as described in example 5. Additionally, to the 2% glycerol batch 60 mM lactose were added initially to the fermentation medium. Continuous lactose feeding with 0.66 M lactose was stared at an OD₆₀₀ₙₘ of about 10. Additionally, lactose supplementation was carried out with a 1 M stock-solution. The lactose concentration was kept at approximately at 30 mM. After leaving the batch phase, indicated by a rise in the dissolved oxygen level, the glycerol feed (60% v/v) started with a flow rate of 6.9 ml/L/h for 37 hours (referring to the starting volume). Afterwards the feed was reduced to 9.4 ml/L/h for 19 hours, and then raised again to 7.3 ml/L/h for 19 hours. 93 hours after seeding the fermenter a 2′-fucosyllactose titer of 150,2 g/L was reached.

### Example 9

Production of 3-Fucosyllactose from Glycerol

Using E. coli BL21 (DE3) lacZ ΔwcaJ ΔfuclK with chromosomal integration of the genes encoding the enzymes for de novo synthesis of GDP-Fucose (ManB, ManC, Gmd, WcaG) a 3-fucosyllactose production strain was constructed.

The gene encoding the alpha-1,3-fucosylltransefrase from Bacteroides fragilis (EP 2439264 A1) together with the gene encoding the sugar efflux transporter SetA from E. coli (US2014/0120611 A1) and a gene conferring gentamycin resistance was integrated into the E. coli genome. Fermentation of the strain to produce 3-fucosyllactose was conducted under conditions described in example 6. Glycerol feeding started after leaving the batch phase with a feeding rate of 7.4 ml/L/h (referring to the starting volume). Lactose was added to the culture to a concentration of 33 mM, when an OD_{600 nm} of 30 was reached. Throughout the process, lactose was added to hold a concentration of at least 10 mM in the supernatant. After 88 h the process was stopped at a 3-fucosyllactose concentration in the supernatant of 30 g/L.

1.-25. (Cancelled)

26. A method for the production of fucosylated oligosaccharides using a genetically modified prokaryotic host cell, the method comprising:
providing a prokaryotic host cell which has been genetically modified to have:
(i) the fructose-6-phosphate pool in the cell increased by increasing the activity of a fructose-1,6-bisphosphate phosphatase;
(ii) overexpression of at least one gene encoding an enzyme necessary for the de novo synthesis of GDP-fucose; and
(iii) expression of an exogenous gene encoding an alpha-1,2-fucosyl-transferase and/or an alpha-1,3-fucosyltransferase; and
cultivating said genetically modified prokaryotic host cell in a cultivation medium comprising at least one carbon and/or energy source selected from one or more of the group consisting of glycerol, succinate, malate, pyruvate, lactate, ethanol, and citrate; and
adding lactose to the cultivation medium;
wherein the fucosylated oligosaccharide is obtained from the medium in which the host cell is cultivated.

27. The method of claim 26, wherein the fucosylated oligosaccharide is selected from the group consisting of 2′-fucosyllactose, 3-fucosyllactose, and difucosyllactose.

28. The method of claim 26, wherein the prokaryotic host cell is selected from the group consisting of bacterial cells from an Escherichia coli strain, a Lactobacillus species, and a Corynebacterium glutamicum strain.

29. The method of claim 26, wherein the phosphomannomutase encoding gene is manB, the mannose-1-phosphate guanosyltransferase encoding gene is manC, the GDP-mannose-4,6-dehydratase encoding gene is gmd, and/or the GDP-L-fucose synthase encoding gene is wcaG.

30. The method of claim 26, wherein the gene encoding the alpha-1,2-fucosyl-transferase is wbgL from E. coli 0126 or fucT2 from Helicobacter pylori.

31. The method of claim 26, wherein the gene encoding the alpha-1,3-fucosyl-transferase is from the species Akkermansia muciniphila, Bacteroides fragilis, Helicobacter pylori, or Helicobacter hepaticus.

32. The method of claim 26, wherein the host cell is further genetically modified to express a gene encoding a protein which enables or facilitates the export of the fucosylated oligosaccharide into the culture medium.

33. The method of claim 32, wherein the gene encoding a protein which enables or facilitates the export of the fucosylated oligosaccharide is a sugar efflux transporter selected from the group consisting of yberc0001_9420 and setA.

34. The method of claim 26, wherein the host cell is further genetically modified to overexpress an endogenous or exogenous permease for the import of lactose.

35. The method of claim 34, wherein the lactose permease is E. coli LacY.

36. The method of claim 26, wherein at least one of the expressed or overexpressed genes in (ii) or (iii) is expressed or overexpressed in a constitutive manner.

37. The method of claim 26, wherein the fructose-1,6-bisphosphate phosphatase is encoded by a gene which is a functional active variant of the fructose-1,6-bisphosphate phosphatase (fbpase) from Pisum sativum.

38. The method of claim 26, wherein the lactose is added from the beginning of the cultivating in a concentration of at least 5 mM, optionally in a concentration of 30, 40, 50, 60, 70, 80, 90, 100, or 150 mM, further optionally in a concentration >300 mM.

39. The method of claim 26, wherein providing of lactose is accomplished by adding lactose to the cultivation medium in a concentration, such that throughout the production phase of the cultivation a lactose concentration is at least 5 mM.

40. The method of claim 39, wherein the lactose concentration is at least 10 mM.

41. The method of claim 39, wherein the lactose concentration is at least 30 mM.

42. The method of claim 26, wherein the host cells are cultivated for at least about 60, about 80, about 100, or about 120 hours or in a continuous manner.

43. The method of claim 26, wherein the modified genes are integrated into the genome of the host strain.

44. The method of claim 26, wherein the at least one gene encoding an enzyme for the de novo biosynthesis of GDP-fucose is a gene encoding a ManA enzyme catalyzing the isomerization of fructose-6-phosphate to mannose-6-phosphate, a phosphomannomutase encoding gene, a mannose-1-phosphate guanosyltransferase encoding gene, GDP-mannose-4,6-dehydratase encoding gene or a GDP-L-fucose synthase encoding gene.

45. The method of claim 26, wherein the genetically modified prokaryotic host cell has further been genetically modified to have reduced or abolished activity of a fructose-6-phosphate-converting enzyme as compared to the activity in an unmodified prokaryotic host cell, wherein the fructose-6-phosphate converting enzyme is selected from the group consisting of phosphofructokinase, glucose-6-phosphate isomerase, fructose-6-phosphate aldolase, a transketolase, and a transaldolase.


SEQUENCE LISTING (8 sequences)

SEQ ID NO: 1 - DNA, 6783 bp, synthetic construct
  source 1..6783: mol_type = other DNA
gccagatgattaattcctaatttttgttgacactctatcattgatagagttattttaccactccctatcagtgatagagaaaagtgaaatgaatagttcgacaaaaatctagaaataattttgtttaactttaagaaggagatatacaatttcgtcgacacacaggaaacatattaaaaattaaaacctgcaggagtttgaaggagatagaaccatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaaaaacgcggccgcgatatcgttgtaaaacgacggccagtgcaagaatcataaaaaatttatttgctttcaggaaaatttttctgtataatagattcataaatttgagagaggagtttttgtgagcggataacaattccccatcttagtatattagttaagtataaatacaccgcggaggacgaaggagatagaaccatgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcggggggggagctaacgcgccatttaaatcaacctcagcggtcatagctgtttcctgtgactgagcaataactagcataaccccttggggcctctaaacgggtcttgaggggttttttgctgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaacgaattaatcttccgcggcggtatcgataagcttgatatcgaattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggtcaaagtagcgatgaagccaacgctcccgttgcagggcagtttgcgcttcccctgagtgccacctttggcttaggggatcgcgtacgcaagaaatctggtgccgcttggcagggtcaagtcgtcggttggtattgcacaaaactcactcctgaaggctatgcggtcgagtccgaatcccacccaggctcagtgcaaatttatcctgtggctgcacttgaacgtgtggcctaatgaggggatcaattctctagagctcgctgatcagaagttcctattctctagaaagtataggaacttcgatggcgcctcatccctgaagccaatagggataacagggtaat

SEQ ID NO: 2 - DNA, 2851 bp, synthetic construct
  source 1..2851: mol_type = other DNA
tggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgctgggagctaagcggccgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacacgcgtgccgcagtctcacgcccggagcgtagcgaccgagtgagctagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcgggtaccggcagggcggggcgtaaggcgcgccatttaaatgaagttcctattccgaagttcctattctctagaaagtataggaactt

SEQ ID NO: 3 - DNA, 2858 bp, synthetic construct
  source 1..2858: mol_type = other DNA
ggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgggcagcattattcgtctgcagggtggtctgggtaatcagctgtttcagtttagctttggttatgccctgagcaaaattaatggtacaccgctgtatttcgacattagccattatgccgaaaacgatgatcatggtggttatcgtctgaataatctgcagattccggaagaatatctgcagtattataccccgaaaattaataatatttataaactgctggtgcgtggcagccgtctgtatccggatatttttctgtttctgggcttttgcaacgaatttcatgcctatggctacgattttgaatatattgcccagaaatggaaaagcaaaaaatacattggctactggcagagcgaacacttttttcataaacatattctggacctgaaagaattttttattccgaaaaatgtgagcgaacaggcaaatctgctggcagcaaaaattctggaaagccagagcagcctgagcattcatattcgtcgtggcgattatattaaaaacaaaaccgcaaccctgacacatggtgtttgtagcctggaatattataaaaaagccctgaacaaaatccgcgatctggcaatgattcgtgatgtgtttatctttagcgacgatatcttctggtgcaaagaaaatattgaaaccctgctgagcaaaaaatataatatttattatagcgaagatctgagccaagaagaggatctgtggctgatgagcctggcaaatcatcatattattgccaatagcagctttagttggtggggtgcatatctgggtagcagcgcaagccagattgttatttatccgaccccgtggtatgatattaccccgaaaaacacctatatcccgattgtgaaccattggatcaacgttgataaacatagcagctgctaagcggccgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacacgcgtcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctcggtaccggcagggcggggcgtaaggcgcgccatttaaatgaagttcctattccgaagttcctattctctagaaagtataggaacttcgaagcagctccag

SEQ ID NO: 4 - DNA, 2631 bp, synthetic construct
  source 1..2631: mol_type = other DNA
ggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaagtcggcactgaccttttcccgtcgcatcaatccggtgtttctggcgttctttgtcgttgcttttctgagcggtatcgcaggcgcactgcaggctccgaccctgagtctgtttctgtccacggaagtgaaagttcgtccgctgtgggttggtctgttctataccgtcaacgcaatcgctggcattacggttagctttatcctggcgaaacgttcagattcgcgcggtgaccgtcgcaagctgattatggtgtgctatctgatggcggttggcaactgtctgctgtttgccttcaatcgtgattacctgaccctgatcacggcaggtgtgctgctggcgagcgttgccaacaccgcaatgccgcagattttcgcgctggcccgtgaatatgccgacagctctgcacgcgaagtggttatgtttagttccatcatgcgcgctcaactgagtctggcatgggtgattggtccgccgctgtcctttatgctggcgctgaattacggttttaccctgatgttctcaatcgcggccggcattttcgttctgtcggccctggtcgtgtggtttatcctgccgagtgtcccgcgtgcagaaccggttgtcgatgcaccggtggttgtccagggttcactgttcgcagacaaaaacgttctgctgctgtttatcgcgtcgatgctgatgtggacctgcaatacgatgtatattatcgatatgccgctgtacattaccgcaagcctgggtctgccggaacgtctggctggtctgctgatgggtaccgcagctggcctggaaattccgatcatgctgctggcgggttattctgtgcgttactttggcaaacgcaagattatgctgttcgctgttctggcgggtgtcctgttttataccggcctggttctgtttaaattcaagacggccctgatgctgctgcagatctttaacgcaattttcatcggtattgtggctggcattggtatgctgtacttccaagatctgatgccgggtcgtgcaggtgcagcaaccacgctgtttaccaatagcatctctacgggtgtcattctggcaggcgtgctgcaaggcggtctgaccgaaacgtggggccatgacagcgtctatgtgatggcgatggtcctgtctattctggccctgattatctgtgcacgtgtgcgcgaagcttaaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttc

SEQ ID NO: 5 - DNA, 4259 bp, synthetic construct
  source 1..4259: mol_type = other DNA
ttactcagcaataaactgatattccgtcaggctggaatactcttcgccaggacgcaggaagcagtccggttgcggccattcagggtggttcgggctgtccggtagaaactcgctttccagagccagcccttgccagtcggcgtaaggttcggttccccgcgacggtgtgccgccgaggaagttgccggagtagaattgcagagccggagcggtggtgtagaccttcagctgcaatttttcatctgctgaccagacatgcgccgccactttcttgccatcgcctttggcctgtaacaagaatgcgtgatcgtaacctttcactttgcgctgatcgtcgtcggcaagaaactcactggcgatgattttggcgctgcggaaatcaaaagacgttccggcgacagatttcaggccgtcgtgcggaatgccgccttcatcaaccggcagatattcgtccgccagaatctgcaacttgtgattgcgcacgtcagactgctcgccgtcaagattgaaatagacgtgattagtcatattcaccgggcaaggtttatcaactgtggcgcgataagtaatggagatacggttatcgtcggtcagacgatattgcaccgtcgcgccgagattacccgggaagccctgatcaccatcatctgaactcagggcaaacagcacctgacgatcgttctggttcacaatctgccagcgacgtttgtcgaacccttccggcccgccgtgcagctggttaacgccctgacttggcgaaagcgtcacggtttcaccgtcaaaggtataacggctattggcgatacggttggcataacgaccaatagaggcccccagaaacgcggcctgatcctgatagcattccgggctggcacagccgagcagcgcctcgcggacgctgccatcggaaagcggaatacgggcggaaagtaaagtcgcaccccagtccatcagcgtgactaccatccctgcgttgttacgcaaagttaacagtcggtacggctgaccatcgggtgccagtgcgggagtttcgttcagcactgtcctgctccttgtgatggtttacaaacgtaaaaagtctctttaatacctgtttttgcttcatattgttcagcgacagcttgctgtacggcaggcaccagctcttccgggatcagcgcgacgatacagccgccaaatccgccgccggtcatgcgtacgccacctttgtcgccaatcacagctttgacgatttctaccagagtgtcaatttgcggcacggtgatttcgaaatcatcgcgcatagaggcatgagactccgccatcaactcgcccatacgtttcaggtcgccttgctccagcgcgctggcagcttcaacggtgcgggcgttttcagtcagtatatgacgcacgcgttttgccacgatcgggtccagttcatgcgcaacagcgttgaactcttcaatggtgacatcacgcagggctggctgctggaagaaacgcgcaccggtttcgcactgttcacgacgggtgttgtattcgctgccaaccagggtacgtttgaagttactgttgatgatgacgacagccacacctttgggcatggaaactgctttggtccccagtgagcggcaatcgatcagcaaggcatgatctttcttgccgagcgcggaaattagctgatccatgatcccgcagttacagcctacaaactggttttctgcttcctgaccgttaagcgcgatttgtgcgccgtccagcggcagatgataaagctgctgcaatacggttccgaccgcgacttccagtgaagcggaagaacttaacccggcaccctgcggcacattgccgctgatcaccatgtccacgccgccgaagctgttgttacgcagttgcagatgtttcaccacgccacgaacgtagttagcccattgatagttttcatgtgcgacaatgggcgcatcgagggaaaactcgtcgagctgattttcataatcggctgccatcacgcgaactttacggtcatcgcgtggtgcacaactgatcacggtttgataatcaatcgcgcagggcagaacgaaaccgtcgttgtagtcggtgtgttcaccaatcaaattcacgcggccaggcgcctgaatggtgtgagtggcagggtagccaaatgcgttggcaaacagagattgtgttttttctttcagactcatttcttacactccggattcgcgaaaatggatatcgctgactgcgcgcaaacgctctgctgcctgttctgcggtcaggtctcgctgggtctctgccagcatttcataaccaaccataaatttacgtacggtggcggagcgcagcagaggcggataaaagtgcgcgtgcagctgccagtgttgattctcttcgccattaaatggcgcgccgtgccagcccatagagtaggggaaggagcactggaagaggttgtcataacgactggtcagctttttcaacgccagcgccagatcgctgcgctgggcgtcggtcaaatcggtgatccgtaaaacgtgggctttgggcagcagtagcgtttcgaacggccaggcagcccagtaaggcacgacggctaaccagtgttcggtttcgacaacggtacggctaccgtctgccagctcgcgctgaacataatccaccagcattggtgatttctgttcggcaaaatattctttttgcaggcggtcttcgcgctcagcttcgttaggcaggaagctatttgcccaaatctgaccgtgcggatgcgggttagagcagcccatcgccgcgcctttgttttcaaaaacctgcacccatgggtacgttttccccagttctgcggtttgctcctgccaggttttgacgatttccgtcaatgctgcaacgctgagctctggcagcgttttactgtgatccggtgaaaagcagatcacccggctggtgccgcgcgcgctctggcaacgcatcagcggatcgtgactttctggcgcatctggcgtgtcagacatcaaagccgcaaagtcattagtgaaaacgtaagtcccggtgtaatcggggtttttatcgcctgtcacccgcacattacctgcgcagaggaagcaatctggatcgtgcgcaggtaacacctgtttggctggcgtttcctgcgccccctgccaggggcgcttagcgcggtgcggtgaaaccagaatccattgcccggtgagcgggttgtagcggcgatgtggatgatcaacgggattaaattgcgtcatggtcgttccttaatcgggatatccctgtggatggcgtgactgccagtgccaggtgtcctgcgccatttcatcgagtgtgcgcgttacgcgccagttcagttcacggtcggctttgctggcgtccgcccagtaggccggaaggtcgccctcgcgacgcggtgcaaaatgataattaaccggtttgccgcaggctttgctgaaggcattaaccacgtccagcacgctgttgcctacgccagcgccgaggttgtagatgtgtacgcctggcttgttcgccagtttttccatcgccacgacgtgaccgtccgccagatccattacgtggatgtaatcgcgtacgccagtaccatcttcggtcggataatcgttaccaaaaatcgccagcgagtcgcgacggcctacagcaacctgggcgatgtatggcatcaggttattcggaatgccttgcggatcttcgcccatatcgcccgacggatgcgcgccaaccgggttgaagtagcgcagcagggcaatgctccagtccggctgggctttttgcagatcggtgaggatctgttccaccatcagcttgcttttgccgtaagggctttgcggtgtgccggtcgggaagctttcaacgtatggaattttgggctgatcgccataaacggtggcggaggagctaaaaataaagtttttgacgttagcggcgcgcatggcgctaatcaggcgcagagtgccgttgacattgttgtcgtaatattccagcggtttttgtaccgattcgcccacggctttcagcccggcgaagtggatcacggtgtcgatagcgtgatcgtgcaggatctcggtcatcaacgcttcgttacgaatatcgccttcaacaaacgttggatgtttgccgcctaaacgctcgataacaggcagtacgctgcgcttactgttacagaggttatcaagaatgatgacatcatgaccgttttgcagtaattgcacacaggtatgacttccaatgtaaccgctaccaccggtaaccagaactctcat

SEQ ID NO: 6 - DNA, 4223 bp, synthetic construct
  source 1..4223: mol_type = other DNA
tggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgcaaaaactactatctttaccgtccaatctggttcagtcttttcatgaactggagagggtgaatcgtaccgattggttttgtacttccgacccggtaggtaagaaacttggttccggtggtggaacatcctggctgcttgaagaatgttataatgaatattcagatggtgctacttttggagagtggcttgaaaaagaaaaaagaattcttcttcatgcgggtgggcaaagccgtcgtttacccggctatgcaccttctggaaagattctcactccggttcctgtgttccggtgggagagagggcaacatctgggacaaaatctgctttctctgcaacttcccctatatgaaaaaatcatgtctttggctccggataaactccatacactgattgcgagtggtgatgtctatattcgttcggagaaacctttgcagagtattcccgaagcggatgtggtttgttatggactgtgggtagatccgtctctggctacccatcatggcgtgtttgcttccgatcgcaaacatcccgaacaactcgactttatgcttcagaagccttcgttggcagaattggaatctttatcgaagacccatttgttcctgatggacatcggtatatggcttttgagtgaccgtgccgtagaaatcttgatgaaacgttctcataaagaaagctctgaagaactaaagtattatgatctttattccgattttggattagctttgggaactcatccccgtattgaagacgaagaggtcaatacgctatccgttgctattctgcctttgccgggaggagagttctatcattacgggaccagtaaagaactgatttcttcaactctttccgtacagaataaggtttacgatcagcgtcgtatcatgcaccgtaaagtaaagcccaatccggctatgtttgtccaaaatgctgtcgtgcggatacctctttgtgccgagaatgctgatttatggatcgagaacagtcatatcggaccaaagtggaagattgcttcacgacatattattaccggggttccggaaaatgactggtcattggctgtgcctgccggagtgtgtgtagatgtggttccgatgggtgataagggctttgttgcccgtccatacggtctggacgatgttttcaaaggagatttgagagattccaaaacaaccctgacgggtattccttttggtgaatggatgtccaaacgcggtttgtcatatacagatttgaaaggacgtacggacgatttacaggcagtttccgtattccctatggttaattctgtagaagagttgggattggtgttgaggtggatgttgtccgaacccgaactggaggaaggaaagaatatctggttacgttccgaacatttttctgcggacgaaatttcggcaggtgccaatctgaagcgtttgtatgcacaacgtgaagagttcagaaaaggaaactggaaagcattggccgttaatcatgaaaaaagtgttttttatcaacttgatttggccgatgcagctgaagattttgtacgtcttggtttggatatgcctgaattattgcctgaggatgctctgcagatgtcacgcatccataaccggatgttgcgtgcgcgtattttgaaattagacgggaaagattatcgtccggaagaacaggctgcttttgatttgcttcgtgacggcttgctggacgggatcagtaatcgtaagagtaccccaaaattggatgtatattccgatcagattgtttggggacgtagccccgtgcgcatcgatatggcaggtggatggaccgatactcctccttattcactttattcgggaggaaatgtggtgaatctagccattgagttgaacggacaacctcccttacaggtctatgtgaagccgtgtaaagacttccatatcgtcctgcgttctatcgatatgggtgctatggaaatagtatctacgtttgatgaattgcaagattataagaagatcggttcacctttctctattccgaaagccgctctgtcattggcaggctttgcacctgcgttttctgctgtatcttatgcttcattagaggaacagcttaaagatttcggtgcaggtattgaagtgactttattggctgctattcctgccggttccggtttgggcaccagttccattctggcttctaccgtacttggtgccattaacgatttctgtggtttagcctgggataaaaatgagatttgtcaacgtactcttgttcttgaacaattgctgactaccggaggtggatggcaggatcagtatggaggtgtgttgcagggtgtgaagcttcttcagaccgaggccggctttgctcaaagtccattggtgcgttggctacccgatcatttatttacgcatcctgaatacaaagactgtcacttgctttattataccggtataactcgtacggcaaaagggatcttggcagaaatagtcagttccatgttcctcaattcatcgttgcatctcaatttactttcggaaatgaaggcgcatgcattggatatgaatgaagctatacagcgtggaagttttgttgagtttggccgtttggtaggaaaaacctgggaacaaaacaaagcattggatagcggaacaaatcctccggctgtggaggcaattatcgatctgataaaagattataccttgggatataaattgccgggagccggtggtggcgggtacttatatatggtagcgaaagatccgcaagctgctgttcgtattcgtaagatactgacagaaaacgctccgaatccgcgggcacgttttgtcgaaatgacgttatctgataagggattccaagtatcacgatcataactgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaagacggccagtgaattcgagctcggtacctaccgttcgtataatgtatgctatacgaagttatcgagctctagagaatgatcccctcattaggccacacgttcaagtgcagcgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcgtagaatttcgacgacctgcagccaagcataacttcgtataatgtatgctatacgaacggtaggatcctctagagtcgacctgcaggcatgagatgtgtataagagacag

SEQ ID NO: 7 - DNA, 3792 bp, synthetic construct
  source 1..3792: mol_type = other DNA
gggaattgattctggtaccaaatgagtcgaccggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgattacccgcaaaaggcgggccaggacaatccatagccgatatccaatcggaatttacgggagcatagtaatgacagatattgcacagttgcttggcaaagacgccgacaaccttttacagcaccgttgtatgactattccttctgaccagctttatctccccggacatgactacgtagaccgcgtgatgattgacaataatcgcccgccagcggtgttacgtaatatgcagacgttgtacaacactgggcgtctggctggcacaggatatctttctattctgccggttgaccagggcgttgagcactctgccggagcttcatttgctgctaacccgctctactttgacccgaaaaacattgttgaactggcgatcgaagcgggctgtaactgtgtggcatcaacttacggcgtgttggcgtcggtatcgcggcgctatgcgcatcgcattccattcctcgtcaaacttaatcacaacgagacgctaagttacccgaacacctacgatcaaacgctgtatgccagcgtggagcaggccttcaacatgggcgcggtggcggttggtgcgactatctattttggttcggaagagtcacgtcgccagattgaagaaatttctgcggcttttgaacgtgcgcacgagctgggcatggtgacagtgctgtgggcctatttgcgtaactccgcctttaagaaagatggcgttgattaccatgtttccgccgacctgaccggtcaggcaaaccatctggcggcgaccataggtgcagatatcgtcaaacaaaaaatggcggaaaataacggcggctataaagcaattaattacggttataccgacgatcgcgtgtacagcaagttaaccagcgaaaacccgattgatctggtgcgttatcagttagctaactgctatatgggccgggccgggttgataaactccggcggtgctgcaggcggtgaaactgacctcagcgatgcagtgcgtactgcggttatcaacaaacgcgctggcggaatggggctgattcttggacgtaaggcgttcaagaaatcgatggctgacggcgtgaaactgattaacgccgtgcaggatgtttatctcgatagcaaaattactatcgcctaagaggatcgagatctcgatcccgcgaaattaatacgactcactataggggaattgtgagcggataacaattcccctctagaaataattttgtttaactttaagaaggagatataccatgggccatcatcatcatcatcatcatcatcatcacagcagcggccatatcgaaggtcgtcatatggcggtgaaagaagcgaccagcgagaccaagaagcgtagcggttacgagatcattaccctgaccagctggctgctgcaacaagaacagaagggtatcattgacgcggaactgaccatcgttctgagcagcattagcatggcgtgcaaacagatcgcgagcctggtgcaacgtgcgaacattagcaacctgaccggtacccaaggcgcggttaacatccagggtgaagaccaaaagaaactggatgttattagcaacgaggtgttcagcaactgcctgcgtagcagcggtcgtaccggcatcattgcgagcgaggaagaggacgtggcggttgcggtggaagagagctacagcggtaactatatcgtggtttttgacccgctggatggcagcagcaacctggatgcggctgtgagcaccggtagcatcttcggcatttacagcccgaacgacgagagcctgccggattttggtgacgatagcgacgataacaccctgggcaccgaagagcaacgttgcatcgttaacgtgtgccaaccgggtagcaacctgctggcggcgggctactgcatgtatagcagcagcgttgcgttcgtgctgaccattggcaagggcgttttcgtgtttaccctggacccgctgtacggtgaattcgtgctgacccaggagaacctgcaaatcccgaagagcggtgaaatttacagctttaacgagggcaactataaactgtgggatgaaaacctgaagaaatatatcgacgatctgaaggaaccgggtccgagcggtaaaccgtacagcgcgcgttatatcggtagcctggttggcgacttccaccgtaccctgctgtacggtggcatttacggttatccgcgtgataagaaaagcaagaacggcaaactgcgtctgctgtatgaatgcgcgccgatgagctttattgttgagcaggcgggtggcaaaggtagcgacggccaccagcgtgtgctggatatccaaccgaccgaaattcaccagcgtgttccgctgtacattggtagcaccgaagaggttgaaaaagttgaaaagtatctggcgtaatcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtggacggccagtgaattcgagctcggtacctaccgttcgtataatgtatgctatacgaagttatcgagctctagagaatgatcccctcattaggccacacgttcaagtgcagcgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcgtagaatttcgacgacctgcagccaagcataacttcgtataatgtatgctatacgaacggtaggatcctctagagtcgacctgcaggc

SEQ ID NO: 8 - DNA, 19 bp, synthetic construct
  source 1..19: mol_type = other DNA
ctgtctcttatacacatct